(19) United States
(12) Patent Application Publication
(10) Pub. No.: US 2022/0168329 A1
(43) Pub. Date: Jun. 2, 2022

(54) COMBINATORIAL DRUG TREATMENT OF CANCER

(71) Applicant: University of Virginia Patent Foundation, Charlottesville, VA (US)

(72) Inventor: Mazhar ADLI, Chicago, IL (US)

(21) Appl. No.: 17/441,984

(22) PCT Filed: Mar. 27, 2020
(86) PCT No.: PCT/US2020/025260

§ 371 Date: Sep. 22, 2021

Related U.S. Application Data
(60) Provisional application No. 62/824,661, filed on Mar. 27, 2019.

(51) Int. Cl. A61K 31/7068; A61K 31/506; A61K 45/06; A61P 35/00

(52) U.S. Cl. CPC A61K 31/7068; A61P 35/00; A61K 45/06; A61K 31/506

(57) ABSTRACT

Described herein are methods for inhibiting protein arginine methyltransferase activity to create conditional vulnerability in tumors, thereby enhancing the effects of DNA damaging agents, methods of inhibiting tumor growth and/or reducing the volume of tumors using the same, and pharmaceutical compositions useful for carrying out the disclosed methods. In a further aspect, the methods and compositions disclosed herein exhibit synthetic lethality to tumor cells but produce few side effects.

### CROSS REFERENCE TO RELATED APPLICATIONS

This application claims priority upon U.S. provisional application Ser. No. 62/824,661 filed on Mar. 27, 2019. This application is hereby incorporated by reference in its entirety.

### STATEMENT REGARDING FEDERALLY SPONSORED RESEARCH OR DEVELOPMENT

This invention was made with government support under grant no. CA211648 awarded by The National Institutes of Health. The government has certain rights in the invention.

### BACKGROUND

Pancreatic ductal adenocarcinoma (PDAC) is the most common and aggressive form of pancreatic cancer. It arises due to abnormal growth of exocrine ductal cells, the digestive enzyme-producing cells that compose 98% of pancreas biomass. PDAC remains one of the deadliest of any cancer type. The main reasons for this high rate of mortality are twofold. First, the disease is mostly asymptomatic until the late stages. Second, current treatment strategies including chemotherapy drug combinations are relatively ineffective. Therefore, surgery, if possible, remains the only curative therapy. However, only 15-20% of PDAC patients are eligible for surgery due to the extension of PDAC to neighboring organs. For the remaining patients, standard treatment involves radiotherapy and chemotherapy combinations. Unfortunately, current chemotherapy combinations have severe side effects due to ineffective selectivity towards PDAC tumors. Thus, the median survival is only about six months, and more than 93% of patients die within the first five years. As such, despite the significant increase in the survival rates of most cancers, PDAC survival remains unchanged in the last fifty years, and it is projected to be the second leading cause of cancer deaths in the USA by 2030. Novel drug combinations that can result in better therapeutic value are desperately needed for PDAC treatment.

Historically, gemcitabine (Gem) has been the first-line chemotherapy and forms the backbone of several drug combinations for the majority of PDAC patients. Gem is a designated “essential medicine” according to the World Health Organization and has been in use since 1983. In addition to being the primary chemotherapy for PDAC, it is a critical therapy in multiple other carcinomas. Although a new multi-drug combination (FOLFIRINOX) slightly improves the survival of PDAC patients, due to high toxicity, only a small fraction of patients tolerate this regimen. Therefore, Gem remains the first-line or second-line for chemotherapy for the majority of PDAC patients. It would be desirable to identify new therapeutic approaches that will enhance the efficacy of DNA damaging agents such Gem when treating cancer in patients such as PDAC.

### SUMMARY

In accordance with the purpose(s) of the present disclosure, as embodied and broadly described herein, the disclosure, in one aspect, relates to methods for inhibiting protein arginine methyltransferase activity to create conditional vulnerability in tumors, thereby enhancing the effects of DNA damaging agents, methods of inhibiting tumor growth and/or reducing the volume of tumors using the same, and pharmaceutical compositions useful for carrying out the disclosed methods. In a further aspect, the methods and compositions disclosed herein exhibit synthetic lethality to tumor cells but produce few side effects.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1F show in vivo CRISPR screening identifies PRMT5 as a novel combinatorial target of gemcitabine (Gem). FIG. 1A shows schematics for in vivo selection screening to identify novel drug combinations. FIG. 1B shows dot plots with gene-specific CRISPR viability scores. Significantly depleted genes (false discovery rate or “FDR”<0.1) are labeled with brown dots, whereas the genes that are deplete both in vitro and in vivo are labeled with blue dots. FIG. 1C shows bar plots with the number of sgRNAs targeting indicated genes among the top 10% of depleted sgRNAs. FIG. 1D shows crystal violet colony formation assays with the relative cell proliferation rates of cells expressing control and PRMT5 targeting sgRNAs in response to the indicated Gem concentrations. FIG. 1E shows dot plots with normalized PRMT5 expression levels in normal and matched PDAC tumors. FIG. 1F shows the Kaplan-Meier plot, demonstrating survival rates of PDAC patients whose tumors have high PRMT5 expression (>0.5 standard deviations) relative to the PRMT5-low patients.

FIGS. 2A-2H show PRMT5 depleted cells are hypersensitive to Gem. FIG. 2A shows a Western blot showing PRMT5 protein levels in wild type (WT) cells, as well as single-cell expanded clones expressing PRMT5 targeting sgRNAs. The β-actin level is shown as a loading control. FIG. 2B shows a line graph with % viability of WT and PRMT5 knockout (KO) PDAC cells in response to increasing doses of Gem. FIG. 2C shows a crystal violet colony formation assay showing the overall survival of indicated WT and PRMT5 KO cells. FIG. 2D shows a crystal violet colony formation assay showing relative growth inhibition activity of two separate PRMT5 inhibitors. FIG. 2E is a Western blot result showing relative levels of symmetric demethylation of arginine (SDMA) in WT cells and PRMT5 KO cells treated with increasing concentrations (nM) of the indicated PRMT5 inhibitor. FIG. 2F is a western blot result showing a relative rate of Caspase-3 cleavage in WT and PRMT5 KO cells treated with increasing doses (nM) of PRMT5 inhibitor. FIG. 2G shows a crystal violet colony formation assay showing relative survival and proliferation rates of mPanc96 (left) and PANC-1 cells (right) treated with various combinatorial doses of Gem and two separate PRMT5 inhibitors. FIG. 2H shows heatmaps representing the Combination Index (CI) values across multiple combinatorial doses in PDX 366T cells. CI<1 indicates synergism.

FIGS. 3A-3G show PRMT5 depletion results in the aberrant transcriptional program of cell cycle and DNA repair genes in response to Gem. FIG. 3A shows MA plots (log fold change vs. log mean expression of each gene) showing the number of differentially regulated genes in WT and PRMT5 KO cells due to Gem treatment. FIG. 3B shows heatmaps and Gene Set Enrichment Analysis (GSEA) showing relative levels of expression changes in genes involved in the indicated cellular processes. FIG. 3C shows flow cytometry cell cycle analysis (DNA content vs. bromodeoxyuridine or BrdU incorporation) of control versus Gem (250 nM) treated WT, PRMT5 KO, or PRMT5 inhibitor EPZ015666 (500 nM) treated cells. The bar plot shows the percentage of cells at the indicated cell cycle stage. ** and *** indicate p values less than 0.01 and 0.001, respectively. FIG. 3D shows Western blots showing relative levels of phosphorylated or total levels of indicated proteins. FIG. 3E shows bar plots of replication protein A2 (RPA2) protein levels quantified from Western blots. FIG. 3F shows Western blots showing levels of RPA1 and RPA2 proteins in PDAC cells treated with various times and doses of the indicated PRMT5 inhibitor. FIG. 3G shows Western blots with relative levels of RPA1 and RPA2 protein levels in WT, PRMT5 KO, and PRMT5 KO cells expressing RPA cDNA. The line plots show the relative viability of indicated cells in response to increasing doses of Gem.

FIGS. 4A-4F show PRMT5 depletion results in impaired DNA repair and excessive DNA damage accumulation in PDAC cells treated with Gem. FIGS. 4A-B show immunofluorescent (IF) images of γ-H2AX (phosphorylated histone 2A family member X) relative levels in WT, PRMT5 KO, and PRMT5 inhibitor EPZ015666 or 938 (500 nM) treated WT mPanc96 cells in response to Gem treatment (250 nM) for the indicated times (upper panels). The dot plots in the lower panel shows quantified IF γ-H2AX levels at the indicated number of single cells. N indicates the number of cells quantified. FIGS. 4C-D show IF images of Comet assay indicating levels of overall DNA strand breaks in WT, PRMT5 KO, and EPZ015666 (500 nM) treated WT mPanc96 cells in response to Gem treatment (250 nM, upper panel). The lower panels show individual cell level quantified length of the comet tail in the indicated number of cells. FIGS. 4E-F show a bar plot with results of I-SceI endonuclease-based genetic reporter assays indicating relative repair efficiency of DNA strand breaks through homology-directed repair (HDR) (FIG. 4E) or non-homology end joining (NHEJ) pathways in HeLa cells treated with Gem (250 nM) and/or EPZ015666 (500 nM) (FIG. 4F).

FIGS. 5A-5F shows genetic depletion or pharmacological inhibition of PRMT5 together results in synergistic tumor growth inhibition with Gem. FIG. 5A is a schematic showing the experimental strategy where WT and PRMT5 KO mPanc96 cells are xenografted in the left and right side of the mice, respectively. The bioluminescence imaging results show relative levels of WT and PRMT5 depleted tumors in control and Gem treated mice. FIG. 5B is a line plot showing caliper-measured relative tumor volumes over time in WT and PRMT5 depleted tumors treated with control and two separate GEM doses. The images show extracted tumors at the end of the experiments. FIG. 5C shows hematoxylin and eosin (H&E) and immunohistochemistry (IHC) stainings show, respectively, tumor architecture and relative levels of PRMT5 protein in tumors originating from WT and PRMT5 KO cells. FIG. 5D shows IHC images and bar plots show relative levels of DNA damage (γ-H2AX staining) in WT and PRMT5 depleted tumors treated with control vehicle or GEM. FIG. 5E shows line plots of caliper-measured relative tumor volumes in vehicle control, single agent or combinatorial Gem, and PRMT5 inhibitor (EPZ015666) treated mice. Pink and blue arrows indicate treatment start times for respective modalities. The images show extracted tumors at the end of the experiment. FIG. 5F shows Western blots indicating relative levels of DNA damage (γ-H2AX) and SDMA in multiple different tumor tissues receiving indicated treatments.

FIG. 6 shows a schematic of a proposed model for PRMT5 depletion mediated impairment of NHEJ DNA repair.

FIG. 7 is a Western blot showing that PRMT5 knockout cells have depleted RPA protein levels compared to WT cells.

FIGS. 8A-8D show that PRMT5 expression is upregulated in PDAC tumors. FIGS. 8A-B are bar plots showing PRMT5 expression levels in normal duct cells, PanIN, or PDAC of human or mouse organoid. FIG. 8C shows dot plots of expression of PRMT5 in individual tumors and stoma cells. FIG. 8D shows PRMT5 expression is inversely correlated with survival of PDAC patients.

FIGS. 9A-9C show dot plots showing PRMT5 levels for TP53 mutant tumors (FIG. 9A) and cancer cell lines (FIG. 9B). FIG. 9C shows plots of overall survival of gemcitabine-treated TP53 WT and mutant PDAC patients with high and low PRMT5 expression levels.

FIG. 10 shows a density plot of sgRNA read count distributions in a Day 0 sample.

FIG. 11 shows a cumulative frequency plot of the fraction of sgRNAs with the indicated number of reads detected in Day 0, untreated tumors, and Gem-treated tumors.

FIG. 12 shows a Venn diagram of the genes represented by 2 or fewer sgRNAs in vitro and in vivo, indicating that their depletion created significant lethality in all samples.

FIG. 13 shows a bar plot of the number of control sgRNAs (total number in library: 360) detected in each of the indicated samples (Day 0 post selection, DMSO in vitro, untreated tumors, Gem in vitro, and Gem-treated tumors).

FIG. 14 shows a Venn diagram of the number of fitness genes detected in vivo and in vitro in the present work and their comparison with previously identified “core fitness” genes from a previously published genome-wide screening.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Aspects of the present disclosure will employ, unless otherwise indicated, techniques of molecular biology, microbiology, organic chemistry, biochemistry, physiology, cell biology, blood vessel biology, and the like, which are within the skill of the art. Such techniques are explained fully in the literature.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a PRMT5 inhibitor,” “a PRMT1 inhibitor,” or “an anti-cancer agent,” includes, but not limited to, mixtures or combinations of two or more such PRMT5 inhibitors, PRMT1 inhibitors, or anti-cancer agents, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “optional” or “optionally” means that the subsequently described event or circumstance can or cannot occur, and that the description includes instances where said event or circumstance occurs and instances where it does not.

As used herein, “protein arginine methyltransferase 5” and “PRMT5” can be used interchangeably, and refer to an enzyme encoded by a gene in humans with a cytogenetic location of 14q11.2 and a molecular location of base pairs 22,920,525 to 22,929,391 on chromosome 14 (Homo sapiens Annotation Release 109, GRCh38.p12). The gene structure in humans includes 17 exons. PRMT5 has an EC classification of 2.1.1.320; an intracellular location within the nucleus or cytoplasm, depending on cell type, differentiation level, and cell cycle stage; and catalyzes the transfer of methyl groups from S-adenosylmethionine to the amino acid arginine in histones, transcriptional elongation factors, and tumor suppressor p53. PRMT5 has also been referred to as histone-arginine-N-methyltransferase 5, protein arginine N-methyltransferase 5, Shk1 kinase-binding protein 1 homolog, 72 kDa ICIn binding protein, Jak-binding protein 1, SKB1 homolog, and HRMT1L5.

As used herein, “protein arginine methyltransferase 1” and “PRMT1” can be used interchangeably, and refer to an enzyme encoded by a gene in humans with a cytogenetic location of 19q13.33 and a molecular location of base pairs 49,676,165 to 49,688,449 on chromosome 19 (Homo sapiens Annotation Release 109, GRCh38.p12). The gene structure in humans includes 12 exons. PRMT1 has an EC classification of 2.1.1.319; an intracellular location within the nucleus and cytoplasm; and catalyzes the transfer of methyl groups from S-adenosylmethionine to the amino acid arginine and is responsible for the majority of cellular arginine methylation activity. PRMT1 has also been referred to as heterogeneous nuclear ribonucleoprotein methyltransferase, histone arginine N-methyltransferase 1, protein arginine N-methyltransferase 1, interferon receptor 1-bound protein 4, HRMT1L2, and IR1B4.

“Guide RNA” or “single guide RNA” (sgRNA) is an RNA that confers target sequence specificity to the CRISPR-Cas9 system. An sgRNA is a short, non-coding sequence that binds to complementary target DNA, allowing Cas9 to perform endonuclease activity in the region of interest.

In one aspect, “symmetric dimethylation of arginine” or “SDMA” occurs when PRMT transfers methyl groups to the terminal guanidine groups of arginine in certain proteins including histones and transcription factors, where methyl groups are added to two separate nitrogen atoms in the guanidine group of arginine. In one aspect, the methyl donor is typically S-adenosyl methionine. In a further aspect, histone arginine methylation is typically associated with gene activation.

In one aspect, “asymmetric dimethylation of arginine” or “ADMA” occurs when PRMT transfers methyl groups to the terminal guanidine groups of arginine in certain proteins including histones and transcription factors, where methyl groups are added to the same nitrogen atom in the guanidine group of arginine. In one aspect, the methyl donor is typically S-adenosyl methionine. In a further aspect, histone arginine methylation is typically associated with gene activation.

“Combination index” (CI) as used herein is a quantitative measure of drug interaction. In one aspect, synergism between two drugs is defined as when CI<1. In one aspect, CI can be calculated using the following equation:

$$CI = \frac{{C}_{A , x}}{{IC}_{x , A}} + \frac{{C}_{B , x}}{{IC}_{x , B}}$$

where A and B represent two drugs to be used in combination, a desired effect for a given drug is represented by IC_{x,A} and IC_{x,B} (for example, IC₅₀), and concentrations of the drugs required to produce the desired effect are represented by C_{A,x} and C_{B,x}.

As used herein, a “heatmap” is a representation of data in graphical form showing values in a matrix as colors. In one aspect, a heatmap can be used as a visual representation of CI values across various combinations of doses of drugs in a particular cell line.

As used herein, “gene set enrichment analysis” or GSEA can be used to identify genes and proteins, or classes thereof, that are overrepresented in a large set. In a further aspect, these overrepresented genes and proteins may be associated with disease phenotypes. In a still further aspect, GSEA is typically accomplished by statistical approaches.

“I-SceI” is an intron-encoded endonuclease typically found in the mitochondria of Saccharomyces cerevisiae. I-SceI recognizes and cuts at TAGGGATAA\hat{ }CAGGGTAAT sites. In one aspect, I-SceI can be used to create double strand breaks, which can then be used in the study of DNA repair mechanisms.

As used herein, “homology directed repair” or HDR is a cellular mechanism for repairing double-strand DNA breaks. In one aspect, homologous recombination is a form of HDR. In a further aspect, HDR can only be used in certain phases of the cell cycle (e.g., G2 and S) where homologous DNA is present in the nucleus.

As used herein, “non-homologous end joining” or NHEJ is a cellular mechanism for repairing double-strand DNA breaks. In one aspect, in NHEJ, these breaks are ligated without the need for a template. In a further aspect, NHEJ uses short, homologous sequences such as, for example, single-stranded overhangs at the ends of double-strand breaks, to guide the DNA repairs. In some aspects, inappropriate NHEJ is commonly found in cancer cells.

As used herein, a “stromal cell” is a connective tissue cell. In some aspects, tumor cells can recruit stromal cells which then provide support for the tumor by mediating therapeutic resistance, promoting angiogenesis, promoting cell proliferation and tissue invasion, and promoting metastasis, among other functions.

“Synthetic lethality” refers to situations in which a combination of deficiencies in the expression of at least two genes leads to cell death, while a deficiency in either of the genes alone does not.

As used herein, “administering” can refer to an administration of the compounds (e.g., compound that inhibits the activity of protein arginine methyl transferase, DNA damaging agent, etc.) and compositions described herein that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components (e.g., compound that inhibits the activity of protein arginine methyl transferase and DNA damaging agent) constituting the kit. Together, the components constitute a functional unit for a given purpose. Individual member components may be physically packaged together or separately. For example, a kit including an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can include one or multiple documents, and are meant to include future updates.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect upon administration of the compounds (e.g., compound that inhibits the activity of protein arginine methyl transferase, DNA damaging agent, etc.) and compositions described herein. The effect can be, but does not necessarily have to be, prophylactic in terms of preventing or partially preventing a disease, symptom or condition thereof, such as, for example, pancreatic ductal adenocarcinoma. The effect can be therapeutic in terms of a partial or complete cure of a disease, condition, symptom or adverse effect attributed to the disease, disorder, or condition. The term “treatment” as used herein can include any treatment in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units of the compounds (e.g., compound that inhibits the activity of protein arginine methyl transferase, DNA damaging agent, etc.) and compositions described herein suitable for use in a subject, each unit containing a predetermined quantity of a pharmaceutical composition thereof calculated to produce the desired response or responses in association with its administration.

The term “solid tumor” as defined herein is an abnormal mass of tissue that usually does not contain cysts or liquid areas. Solid tumors may be benign (not cancer), or malignant (cancer). Different types of solid tumors are named for the type of cells that form them. Examples of solid tumors are sarcomas, carcinomas, and lymphomas.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of the compounds (e.g., compound that inhibits the activity of protein arginine methyl transferase, DNA damaging agent, etc.) and compositions described herein provided herein that is sufficient to effect beneficial or desired biological, emotional, medical, or clinical response of a cell, tissue, system, animal, or human. An effective amount can be administered in one or more administrations, applications, or unit dosages. The term can also include within its scope amounts effective to enhance or restore to substantially normal physiological function.

As used herein, the term “therapeutically effective amount” refers to an amount of the compounds (e.g., compound that inhibits the activity of protein arginine methyl transferase, DNA damaging agent, etc.) and compositions described herein that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of the compounds (e.g., compound that inhibits the activity of protein arginine methyl transferase, DNA damaging agent, etc.) and compositions described herein, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a pharmaceutical composition, by changing the pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount of the compounds (e.g., compound that inhibits the activity of protein arginine methyl transferase, DNA damaging agent, etc.) and compositions described herein effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C1-to-C6 alkyl esters and C5-to-C7 cycloalkyl esters, although C1-to-C4 alkyl esters are preferred. Esters of gemcitabine, PRMT5 inhibitors, and/or PRMT1 inhibitors can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C1-to-C6 alkyl amines and secondary C1-to-C6 dialkyl amines. In the case of secondary amines, the amine can also be in the form of a 5- or 6-membered heterocycle containing one nitrogen atom. Amides derived from ammonia, C1-to-C3 alkyl primary amides and C1-to-C2 dialkyl secondary amides are preferred. Amides of gemcitabine, PRMT5 inhibitors, and/or PRMT1 inhibitors can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of gemcitabine, a PRMT5 inhibitor, and/or a PRMT1 inhibitor, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

Unless otherwise specified, temperatures referred to herein are based on atmospheric pressure (i.e. one atmosphere).

Methods for Inhibiting Tumor Growth and/or Reducing Tumor Volume

In one aspect, disclosed herein is a method for inhibiting tumor growth in a subject, the method including the steps of administering to the subject (1) a compound that inhibits a protein arginine methyl transferase (PRMT) and (2) a DNA damaging agent. In one aspect, the tumor can be a brain tumor, ovarian tumor, prostate tumor, or breast tumor. In another further aspect, the tumor can be a pancreatic ductal adenocarcinoma or other pancreatic tumor. In one aspect, inhibiting tumor growth can involve slowing the rate of growth of the tumor or completely stopping the growth of a tumor relative to a control, wherein the control can be a tumor which is not treated or to which only the compound that inhibits PRMT or the DNA damaging agent is administered, but not both. In some aspects, the compound inhibits PRMT5 activity. In other aspects, the compound inhibits PRMT1 activity. In one aspect, a compound that inhibits PRMT5 activity and a compound that inhibits PRMT1 activity can both be administered. In one aspect, the method reduces tumor growth from about 10% to about 100% relative to a control, or about 10%, about 20%, about 30%, about 40%, about 50%, about 60%, about 70%, about 80%, about 90%, or about 100%, where any value can be a lower and upper endpoint of range (e.g., about 30% to about 80%, etc.).

In another aspect, disclosed herein is a method for reducing the volume of a tumor in a subject, the method including the steps of administering to the subject (1) a compound that inhibits a protein arginine methyl transferase and (2) a DNA damaging agent. In some aspects, the compound inhibits PRMT5 activity. In other aspects, the compound inhibits PRMT1 activity. In one aspect, a compound that inhibits PRMT5 activity and a compound that inhibits PRMT1 activity can both be administered. In one aspect, reducing tumor volume can involve slowing the rate of growth of the tumor or completely stopping the growth of a tumor relative to a control, wherein the control can be a tumor which is not treated or to which only the compound that inhibits PRMT or the DNA damaging agent is administered, but not both. In one aspect, the method reduces tumor volume from about 10% to about 100% relative to a control, or about 10%, about 20%, about 30%, about 40%, about 50%, about 60%, about 70%, about 80%, about 90%, or about 100%, where any value can be a lower and upper endpoint of range (e.g., about 30% to about 80%, etc.).

In yet another aspect, disclosed herein is a method for inhibiting the level of symmetric arginine demethylation (SDMA), asymmetric arginine demethylation (ADMA), or a combination thereof of a protein in tumor cells in a subject, the method including the steps of administering to the subject (1) a compound that inhibits the activity of a PRMT and (2) a DNA damaging agent. In one aspect, reducing SDMA or ADMA level can involve slowing the rate of growth of the tumor or completely stopping the SDMA or ADMA relative to a control, wherein the control can be a tumor which is not treated or to which only the compound that inhibits PRMT or the DNA damaging agent is administered, but not both. In some aspects, the compound inhibits PRMT5 activity. In other aspects, the compound inhibits PRMT1 activity. In one aspect, a compound that inhibits PRMT5 activity and a compound that inhibits PRMT1 activity can both be administered. In one aspect, the method inhibits the level of symmetric arginine demethylation (SDMA), asymmetric arginine demethylation (ADMA), or a combination thereof in tumor cells from about 10% to about 100% relative to a control, or about 10%, about 20%, about 30%, about 40%, about 50%, about 60%, about 70%, about 80%, about 90%, or about 100%, where any value can be a lower and upper endpoint of range (e.g., about 30% to about 80%, etc.).

In yet another aspect, disclosed herein is a method for increasing the activation level of γ-H2AX in tumor cells in a subject, the method including the steps of administering to the subject (1) a compound that inhibits the activity of a PRMT and (2) a DNA damaging agent. In one aspect, increasing the activation level of γ-H2AX is relative to a control, wherein the control can be a tumor which is not treated or to which only the compound that inhibits PRMT or the DNA damaging agent is administered, but not both. In some aspects, the compound inhibits PRMT5 activity. In other aspects, the compound inhibits PRMT1 activity. In one aspect, a compound that inhibits PRMT5 activity and a compound that inhibits PRMT1 activity can both be administered. In one aspect, the method increases the activation level of γ-H2AX in tumor cells from about 10% to about 100% relative to a control, or about 10%, about 20%, about 30%, about 40%, about 50%, about 60%, about 70%, about 80%, about 90%, or about 100%, where any value can be a lower and upper endpoint of range (e.g., about 30% to about 80%, etc.).

Compounds that Inhibit the Activity of Protein Arginine Methyl Transferase (PRMT)

The methods described herein involve the administration of a compound that inhibits the activity of a protein arginine methyl transferase (PRMT). In one aspect, the compound inhibits the level of symmetric arginine dimethylation, asymmetric arginine dimethylation, or a combination thereof of one or more proteins in tumor cells in a subject compared to the level of symmetric arginine dimethylation, asymmetric arginine dimethylation, or a combination thereof in the tumor cells of the same subject that is not administered the compound.

In one aspect, the compound inhibits the activity of protein arginine methyl transferase 5 (PRMT5). Exemplary PRMT5 inhibitors are shown below:

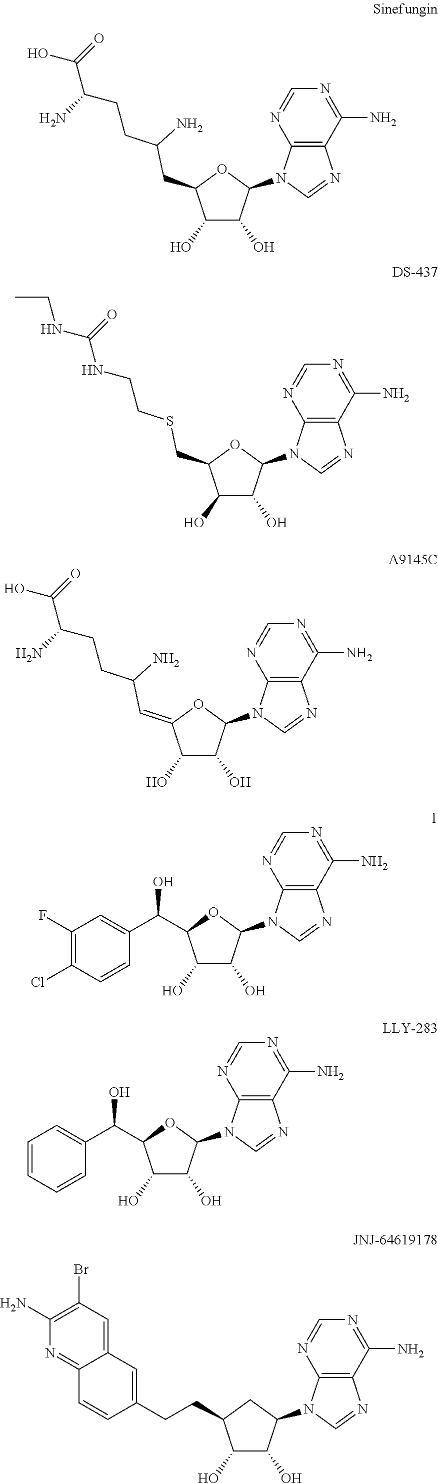 [SMILES: Nc1ncnc2c1ncn2[C@@H]1O[C@H](CC(N)CC[C@H](N)C(=O)O)[C@@H](O)[C@H]1O ; CCNC(=O)NCCSC[C@H]1O[C@@H](n2cnc3c(N)ncnc32)[C@H](O)[C@H]1O ; Nc1ncnc2c1ncn2[C@@H]1O/C(=C\C(N)CC[C@H](N)C(=O)O)[C@@H](O)[C@H]1O ; Nc1ncnc2c1ncn2[C@@H]1O[C@H]([C@H](O)c2ccc(Cl)c(F)c2)[C@@H](O)[C@H]1O ; Nc1ncnc2c1ncn2[C@@H]1O[C@H]([C@H](O)c2ccccc2)[C@@H](O)[C@H]1O ; Nc1nc2ccc(CC[C@H]3C[C@@H](n4cnc5c(N)ncnc54)[C@H](O)[C@@H]3O)cc2cc1Br]

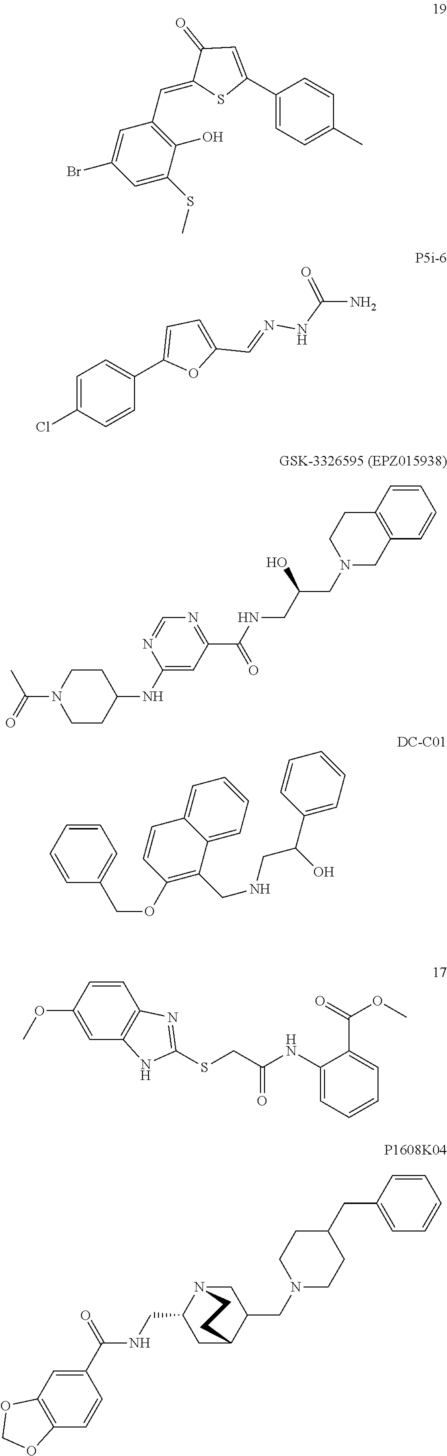

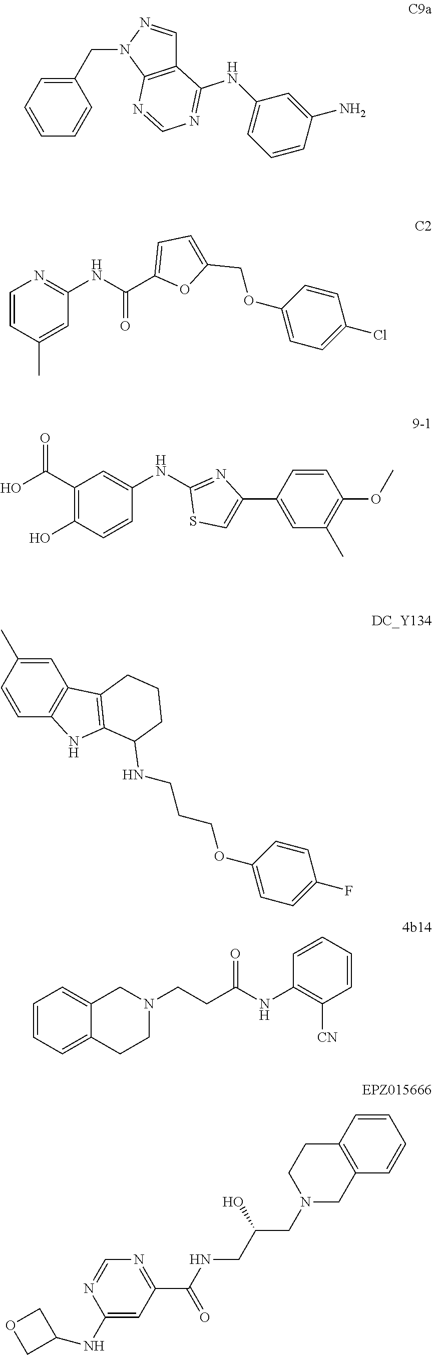

In one aspect, the PRMT5 inhibitor is JNJ-64619178, EPZ015666, EPZ015938, PF-06939999, or a combination thereof.

In another aspect, the compound inhibits the activity of protein arginine methyl transferase 1 (PRMT1). Exemplary PRMT1 inhibitors are shown below:

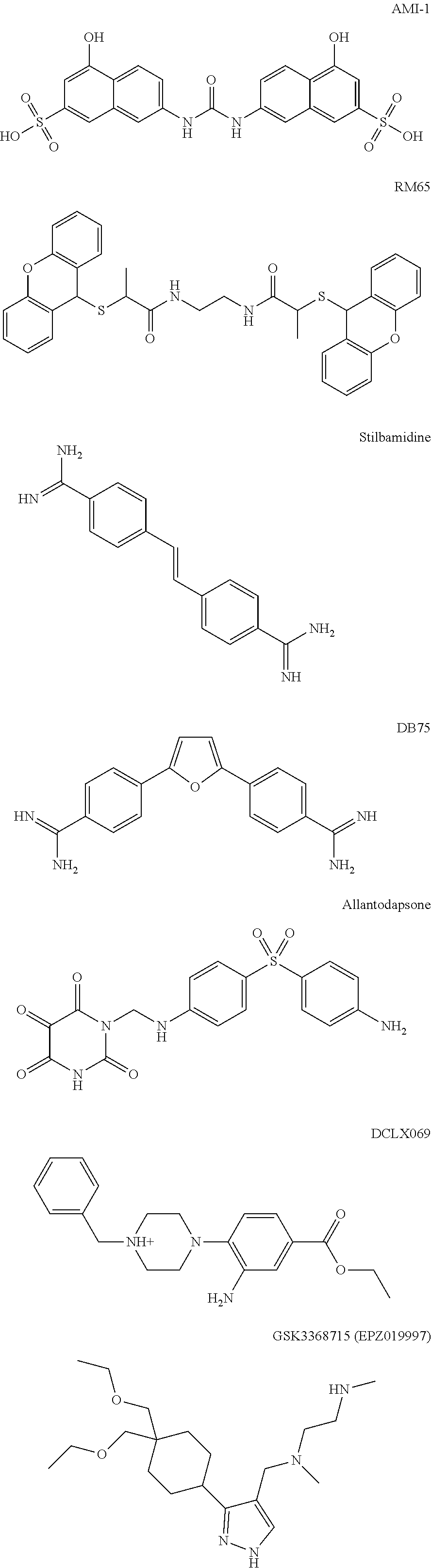

In some aspects, inhibitors for both PRMT5 and PRMT1 can be used sequentially or simultaneously in the methods disclosed herein.

### DNA Damaging Agent

The DNA damaging agent is a compound that can facilitate the repair of damaged DNa such as, for example, an alteration in the chemical structure of DNA, such as a break in a strand of DNA, a base missing from the backbone of DNA, or a chemically changed base. In another aspect, gemcitabine, or 2′,2′-difluoro 2′-deoxycytidine, or dFdC, is a chemotherapy medication useful in the treatment of various cancers including, but not limited to, breast cancer, ovarian cancer, non-small cell lung cancer, pancreatic cancers including pancreatic ductal adenocarcinoma, and/or bladder cancer.

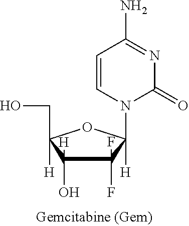

In a further aspect, the compositions disclosed herein include a therapeutically-effective amount of gemcitabine. Gemcitabine is well-tolerated in many patients. Without wishing to be bound by theory, gemcitabine can be transported into cells in the same manner as other nucleosides and is then phosphorylated at the 5′ position by several different enzymes to become dFdCTP, where it can mimic deoxycytidine triphosphate and be incorporated into new DNA, wherein it evades DNA repair enzymes in the cell while leading to inhibition of further DNA synthesis.

In another aspect, the DNA damaging agent can be doxorubicin, cisplatin, carboplatin, oxaliplatin, picoplatin, methotrexate, daunorubicin, 5-fluorouracil, capecitabine, floxuridine, 6-mercaptopurine, 8-azaguanine, fludarabine, cladribine, aminopterin, ralitrexed, etoposide, teniposide, campothecin, doxorubicin, epirubicin, idarubicin, or a combination thereof.

### Dosages and Administration

In one aspect, the PRMT inhibitor can be administered to a patient in a dosage of from about 0.1 mg to about 8 mg per day, or at about 0.1 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg per day, or a combination of any of the foregoing values, or a range encompassing any of the foregoing values (e.g., about 3 mg to about 9 mg, etc.).

In one aspect, the DNA damaging agent is administered in a dosage of about 100 mg/m² to about 2,000 mg/m² of body surface area, or about 100 mg/m², about 200 mg/m², about 300 mg/m², about 400 mg/m², about 500 mg/m², about 600 mg/m², about 700 mg/m², about 800 mg/m², about 900 mg/m², about 1,000 mg/m², about 1,100 mg/m², about 1,200 mg/m², about 1,300 mg/m², about 1,400 mg/m², about 1,500 mg/m², about 1,600 mg/m², about 1,700 mg/m², about 1,800 mg/m², about 1,900 mg/m², or about 2,000 mg/m², or a combination of any of the foregoing values, or a range encompassing any of the foregoing values (e.g., about 800 mg/m² to about 1,200 mg/m², etc.). In another aspect, the DNA damaging agent can be gemcitabine administered in a dosage of about 800 mg/m² to about 1,400 mg/m² of body surface area, or about 800 mg/m², about 900 mg/m², about 1,000 mg/m², about 1,100 mg/m², about 1,200 mg/m², about 1,300 mg/m², about 1,400 mg/m², or a range encompassing any of the foregoing values (e.g., about 800 mg/m² to about 1,200 mg/m², etc.).

The compound that inhibits PRMT activity and the DNA damaging agent can be administered as neutral compounds or pharmaceutically acceptable salts thereof. Bases that can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

Acids which can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt can include an anion formed from hydrobromic, hydrochloric, maleic, phosphoric, sulfuric, and tartaric acids.

In another aspect, the compound that inhibits PRMT activity and the DNA damaging agent can be administered as pharmaceutically acceptable esters, prodrugs, hydrates, solvates, or polymorphs thereof.

In one aspect, the compound that inhibits the activity of a PRMT can be administered prior to the administration of the DNA damaging agent. In an alternative aspect, the compound that inhibits the activity of a PRMT is administered concurrently with the DNA damaging agent. In one aspect, the compound that inhibits the activity of a PRMT and the DNA damaging agent are packaged in two separate dosage forms. In another aspect, the compound that inhibits the activity of a PRMT and the DNA damaging agent are packaged in a single dosage form. In some aspects, the compound that inhibits the activity of a PRMT and the DNA damaging agent are administered a single time. In other aspects, the compound that inhibits the activity of a PRMT and the DNA damaging agent are administered multiple times.

In certain aspects, the compound that inhibits the activity of a PRMT and DNA damaging agent can be formulated with a pharmaceutically-acceptable carrier to produce a pharmaceutical composition to be administered to the subject. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions contain a therapeutically effective amount of a DNA damaging agent or a pharmaceutically acceptable salt thereof, a compound that inhibits PRMT activity or a pharmaceutically acceptable salt thereof, a pharmaceutically acceptable carrier, optionally one or more other therapeutic agents, and optionally one or more adjuvants. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitonealy, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

### Kits

In a further aspect, the present disclosure relates to kits including (1) a compound that inhibits the activity of protein arginine methyl transferase (PRMT) and (2) a DNA damaging agent. The kit can also include instructions for administering the compounds.

The a compound that inhibits the activity of protein arginine methyl transferase (PRMT) and a DNA damaging agent can conveniently be presented as a kit, where the compound that inhibits the activity of protein arginine methyl transferase (PRMT) and the DNA damaging agent, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compound that inhibits the activity of protein arginine methyl transferase (PRMT) and the DNA damaging agent. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

In a further aspect, the present disclosure also provides a pharmaceutical pack or kit that includes one or more containers filled individually with the compound that inhibits the activity of protein arginine methyl transferase (PRMT) and the DNA damaging agent. Associated with such container(s) can be a notice in the form prescribed by a governmental agency regulating the manufacture, use or sale of pharmaceuticals or biological products, which notice reflects approval by the agency of manufacture, use or sale for human administration.

It is contemplated that the disclosed kits can be used in connection with the disclosed methods of making, the disclosed methods of using or treating, and/or the disclosed compositions.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

Exemplary Aspects

Aspect 1: A method for inhibiting tumor growth in a subject comprising administering to the subject (1) a compound that inhibits the activity of a protein arginine methyl transferase (PRMT) and (2) and a DNA damaging agent.

Aspect 2: A method for reducing the volume of a tumor in a subject comprising administering to the subject (1) a compound that inhibits the activity of a protein arginine methyl transferase (PRMT) and (2) and a DNA damaging agent.

Aspect 3: A method for inhibiting the level of symmetric arginine dimethylation, asymmetric arginine dimethylation, or a combination thereof of a protein in tumor cells in a subject comprising administering to the subject (1) a compound that inhibits the activity of a protein arginine methyl transferase (PRMT) and (2) and a DNA damaging agent.

Aspect 4: A method for increasing the activation level of γ-H2AX in tumor cells in a subject comprising administering to the subject (1) a compound that inhibits the activity of a protein arginine methyl transferase (PRMT) and (2) and a DNA damaging agent.

Aspect 5: The method in any one of aspects 1 to 4, wherein compound inhibits the activity of protein arginine methyl transferase 1 (PRMT1).

Aspect 6: The method in any one of aspects 1 to 4, wherein compound inhibits the activity of protein arginine methyl transferase 5 (PRMT5).

Aspect 7: The method in any one of aspects 1 to 4, wherein compound inhibits the activity of protein arginine methyl transferase 1 and 5.

Aspect 8: The method in any one of aspects 1 to 7, wherein the compound inhibits the activity of protein arginine methyl transferase 1 comprises GSK3368715, AMI-1, RM65, DB75, stilbamidine, alantodapsone, DCLX069, or any combination thereof.

Aspect 9: The method in any one of aspects 1 to 7, wherein the compound inhibits the activity of protein arginine methyl transferase 5, wherein the compound is EPZ015666, EPZ015938, JNJ-64619178, PF-06939999, or any combination thereof.

Aspect 10: The method in any one of aspects 1 to 9, wherein the compound that inhibits the activity of protein arginine methyl transferase is administered at a dosage of from about 0.1 mg to about 10 mg/day.

Aspect 11: The method in any one of aspects 1 to 10, wherein the DNA damaging agent comprises gemcitabine, doxorubicin, cisplatin, carboplatin, oxaliplatin, picoplatin, methotrexate, daunorubicin, 5-fluorouracil, capecitabine, floxuridine, 6-mercaptopurine, 8-azaguanine, fludarabine, cladribine, aminopterin, ralitrexed, etoposide, teniposide, campothecin, doxorubicin, epirubicin, idarubicin, or any combination thereof.

Aspect 12: The method in any one of aspects 1 to 10, wherein the DNA damaging agent is gemcitabine is administered at a dosage of from about 800 mg/m² to about 1,400 mg/m².

Aspect 13: The method in any one of aspects 1 to 10, wherein the compound inhibits the activity of protein arginine methyl transferase 5 (PRMT5) and the DNA damaging agent is gemcitabine.

Aspect 14: The method in any one of aspects 1 to 13, wherein the compound that inhibits the activity of protein arginine methyl transferase is administered prior to the administration of the DNA damaging agent.

Aspect 15: The method in any one of aspects 1 to 14, wherein the tumor comprises a solid tumor.

Aspect 16: The method in any one of aspects 1 to 14, wherein the subject has brain cancer, ovarian cancer, prostate cancer, breast cancer, or pancreatic cancer.

Aspect 17: The method in any one of aspects 1 to 14, wherein the subject has pancreatic ductal adenocarcinoma (PDAC).

Aspect 18: A kit comprising (1) a compound that inhibits the activity of a protein arginine methyl transferase (PRMT) and (2) and a DNA damaging agent.

Aspect 19: A pharmaceutical composition comprising (1) a compound that inhibits the activity of a protein arginine methyl transferase (PRMT) and (2) and a DNA damaging agent.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Materials and Methods

### In Vitro Cell Culture

Human PDX366 (Patient-derived pancreatic tumor cells), mPanc96, and PANC-1 pancreatic carcinoma cells were cultured in RPM11640 medium supplemented with 10% fetal bovine serum (FBS) and 1% streptomycin/penicillin. Cells were treated with gemcitabine (GEMZAR; Eli Lilly) and/or either EPZ015666 (GSK3235025; SelleckChem) or EPZ015938 (GSK3326595; ChemieTek).

Generation of CRISPR sgRNA Library Pool and Viral Infection

PDX366 cell line was produced from pancreatic patients. WT Cas9 expressing lentivirus was generated in HEK293T cell line by co-transfection of WT Cas9 (modified from GeCKO plasmid by removing gRNA), psPAX and pMD2.6 plasmid with 5:4:1 ratio. 10 μg total DNA was used in the presence of 30 μL of Fugene6 reagent in a 10-cm plate dish that had 70% confluency. PDX366 cell line was infected with this lentivirus for one day and then treated with 0.5 μg/ml puromycin for four days. The nuclear sgRNA libraries were kind gifts from the Sabatini lab (MIT). The libraries were amplified using a protocol provided by the manufacturer. The library pool targets 619 epigenetic regulators with ˜ 10 sgRNA/gene. 360 non-genomic targeting control sgRNAs are included in the library. The sgRNA library expressing viruses was generated in 2×15 cm plates by using a total of 20 μg DNA and the condition mentioned above. Serial dilutions of a virus were used to find the MOI of ˜0.25 after selection with five μg/mL blasticidin for 4 days. Cells were harvested from 12×15 cm plates to get at least 200× fold coverage (˜2 million cells per sample) for the in vitro and in vivo (orthotopic injection into mouse pancreas) screening.

### In Vivo CRISPR Screening in an Orthotopic Patient-Derived Xenograft (PDX) Model of PDAC

6-7 week-old athymic nude mice (Envigo, Indianapolis, Ind.) were used for in vivo screening and selection. The sgRNA library WT Cas9 expressing PDX366 cells were resuspended in 150 μL MATRIGEL® Growth Factor Reduced Basement Membrane Matrix (Corning, Corning, N.Y.). After anesthesia, the left flank of the mouse was opened to exteriorize the pancreas, and 8×10⁶ PDX366 cells were injected directly into the pancreas. At this stage, one batch of cells was harvested as “day 0” control sample. For in vitro screening, cells were passaged every 3-4 days by 1:3 split with fresh media in 15 cm plates. At least 12 million cells were passaged each time using 3×15 cm plates.

Tumor volumes were monitored by magnetic resonance imaging (MRI). MRI measurement (University of Virginia Molecular Imaging Core, Charlottesville, Va.) was performed after four weeks, at the conclusion of the experiment. Tumors were harvested and weighed, and samples collected for further analysis. Formalin-fixed tumor samples were submitted to the University of Virginia Research Histology Core Lab for processing and H&E staining. Tumor sections were scored by a board-certified pathologist who specializes in gastrointestinal cancers. This study was carried out in strict accordance with the recommendations in the Guide for the Care and Use of Laboratory Animals of the National Institutes of Health. The animal protocol was approved by the Animal Care and Use Committee of the University of Virginia.

### Targeted Amplification of CRISPR/sgRNA Library and Sequencing

Tumors from mice and in vitro cultured cells were harvested after four weeks. Entire tumors and all cell pellets were used to obtain genomic DNA. Briefly, tumor samples were minced into small pieces and lysed with 8 mL SDS lysis buffer (100 mM NaCl, 50 mM Tris-Cl pH 8.1, 5 mM EDTA, and 1% wt/vol SDS). Cell pellets were processed in a similar way. Minced tumor samples or cell pellets were treated with 100 μL proteinase K (20 mg/ml) at 55° C. for overnight incubation. The next day, entire lysis solutions were used in EtOH precipitation, and genomic DNA pellets washed with 70% EtOH twice. Pellets were resuspended in RNase-containing water and quantified by Nanodrop. For each DNA sample, 100 μg genomic DNA was used for the first PCR reaction. We ran ten separate PCR reactions with ten μg DNA in a single PCR tube. We used the same outer Forward Primer and outer Reverse Primer from Sabatini sgRNA library-specific primers for all of the samples (these primers are different from GeCKO Array For and Rev). Q5-high Fidelity 2× master mix was used as polymerase from NEB (#M0429L). PCR condition for the first PCR was; 98° C. for 30 sec, 18× (98° C. for 10 sec, 63° C. for 10 sec, 72° C. for 25 sec), 72° C. for 2 min. After the first PCR, all reactions were combined (10×100 μL) in one single Eppendorf tube and vortexed well. For the second PCR, 5 μL PCR reaction mix from the first PCR step was used in 100 μL total PCR reaction. PCR conditions for the second PCR were: 98° C. for 30 sec, 24× (98° C. for 10 sec, 63° C. for 10 sec, 72° C. for 25 sec), 72° C. for 2 min. In the second PCR, each sample was amplified with specific forward primers that had a six bp barcode sequence for demultiplexing of our reads during next-generation sequencing and common reverse primer. In this setting, custom sequencing and custom indexing primers for Illumina Sequencing were used. The entire solution from the second PCR was loaded on a 2% gel, and the bands around 270 bp were cut and cleaned with the Qiagen gel extraction kit (a faint band above 270 bp was noticed, likely due to carrying over of primers from the first PCR reaction). Purified PCR products were quantified by using Qubit (Invitrogen), and equimolar amounts of each PCR fragment were mixed and used for subsequent high-throughput sequencing (40 nM DNA in 20 μL). The library was sequenced using the Illumina Miseq platform to get an average of 10 million reads for each sample.

### Data Analysis for CRISPR/Cas9 Screening

Sequencing reads from CRISPR/Cas9 screenings were first demultiplexed with cutadapt (v. 1.8.3). Sequences of a total length of 56 nt (sequencing barcode and sample barcode) were supplied to the program with the requirements that at least 36 nt of this barcode had to be present in the read, so that it could be assigned to an individual tumor isolated from the PDX model. More than 99% of reads were assigned to one of the three in vitro and six in vivo samples: cells from the day of injection (further referred to as day 0), control and gem treated in vitro samples (one each), and control and gem treated in vivo samples (3 each). After de-multiplexing and removing sequencing and sample barcodes, the abundance of each sgRNA was assessed and normalized among samples with the use of MAGeCK v. 0.5.2. About 87% of the reads contained correct sgRNA sequences.

Downstream data analysis was performed in RStudio v. 0.99.484 with R v. 3.3.0 following a published procedure with slight modifications. We performed the following analysis to identify potential combinatorial targets of gemcitabine. The first step of this analysis was to calculate the relative abundance of sgRNAs targeting each gene between “day 0” and one of the other eight samples by comparing normalized average counts of all the sgRNAs targeting the particular gene. Since the non-genomic targeting control sgRNAs were well represented in all the samples, they were used to profile the null distribution of Robust Rank Aggregation (RRA) scores when calculating the P values. Based on the negative selection RRA scores, one of the in vivo gem treated samples had a substantially higher sgRNA depletion rate compared to the other two replicates, and thus was excluded from the downstream analysis. Genes consistently depleted in all the samples compared to “day 0” were likely to be essential genes for the PDX cell line, and were removed from the downstream analysis. The second step of the analysis was to calculate log fold change (LFC) of mean read counts between gem treated and control samples for all the retained genes in in vitro and in vivo settings, respectively. In the third step, we ranked all the retained genes based on LFC, and genes significantly depleted (FDR q<0.1) in both in vitro and in vivo screenings were selected as candidate combinatorial targets of gemcitabine.

### Validation of PRMT5 as a Viable CRISPR Screening Hit

For validation of PRMT5 after the initial screening, the following sgRNA target sequences (sgRNAs) were designed and cloned to generate PRMT5 knock out cells:

sgRNA1:
GGTACCCTTGGTGGCACCAG
sgRNA2:
GGTGATGGCCAGTGTGGATG
sgRNA3:
GTAAGGGGCAGCAGGAAAGC

Briefly, the oligos that have -5′CACC and -5′AAAC overhangs of the sgRNA guiding sequence were ordered from Eurofins and hybridized to get sticky end double-strand DNA for ligation. The plasmid containing the sgRNA backbone was digested with Bbs.i at 55° C. for 2 hours, followed by CIP treatment at 37° C. for 30 min. Purified vector backbone from a 2% gel (60 ng) and hybridized oligos (1 μL from 1-10 nM) were used for the ligation reaction in the presence of T4 ligase.

WT Cas9 and gRNA expressing lentivirus were generated using the HEK293T cell line. mPanc96 and PANC-1 cells were virally infected to express Cas9 and sgRNA to produce stable cell lines. After four days of puromycin selection (2 μg/mL), serial dilution was performed to generate single clones. Once the desired number of clones was obtained, lysates were prepared in RIPA buffer, and Western Blot was performed to determine PRMT5 knockout efficiency.

### MTT Cell Viability

PDX366, mPanc96, and PANC-1 cells were seeded in a flat-bottom 96-well plate (Corning) in triplicate at a density of 1-2×10³ cells per well. The following day, cells were treated with gemcitabine (GEMZAR; Eli Lilly) and EPZ015666 (GSK3235025; SelleckChem) or EPZ015938 (GSK3326595; ChemieTek) for 4-5 days prior to MTT (3-(4,5-dimethylthiazolyl)-2,5-diphenyltetrazolium bromide) to determine effects of drugs on cell viability. Culture media were replaced with fresh RPMI, which had 10% FBS and 10% MTT (5 mg/mL) and incubated for 4 hours in a humidified (37° C., 5% CO₂) incubator. 100 μL MTT solvent (10% SDS in 0.01 M HCl) was added to each well, and cells were incubated overnight. The absorbance was read at 595 nm.

### Crystal Violet Assay

Pancreatic cancer cells were seeded in a flat-bottom 12-well plate (Corning) at a density of 1-2×10³ cells per well. The following day, cells were treated with gemcitabine and EPZ015666 or EPZ015938 for two weeks. Culture media were replaced every week with fresh medium in the presence of drugs. Wells were washed with PBS, then stained for 30 min with crystal violet solution (0.4% crystal violet, 10% formaldehyde, 80% methanol). After staining, wells were washed once with PBS and water. The plate was dried out overnight and imaged using a scanner. Colonies were measured and analyzed with ImageJ (National Institutes of Health).

### Annexin V Staining

Annexin V staining was performed to determine the percentage of apoptotic cells. After treatment with gemcitabine and EPZ015666 or EPZ015938, the pancreatic cancer cells were washed with cold PBS, resuspended in Annexin V binding buffer (10 mM HEPES, 140 mM NaCl, and 2.5 mM CaCl₂, pH 7.4) with an appropriate amount of FITC-conjugated Annexin V antibody (Life Technologies #A13199), and incubated at room temperature (RT) for 15 min. After washing with binding buffer, the cells were resuspended in 2 μg/mL propidium iodide (PI) (Sigma) in PBS plus RNase, incubated at RT for 15 min in the dark, and then analyzed using a FACSCalibur flow cytometer (Becton-Dickinson, San Jose, Calif., USA).

### BrdU Staining

Pancreatic cancer cells were treated with gemcitabine and EPZ015666 or EPZ015938, incorporated with BrdU (Sigma) for 1 hour and then fixed by 70% ethanol. BrdU staining was performed according to the manufacturer's instructions (BD Biosciences, Franklin Lakes, N.J., USA). Briefly, the fixed cells were washed with PBS and then resuspended in 2 N HCl for 20 min to denature the DNA. After washing with 0.1 M Na₂B₄O₇, pH 8.5, to stop acid denaturation, the cells were resuspended and washed with 180 μL 0.5% polysorbate 20 (Sigma) with 1% normal goat serum (NGS) (Dako, Glostrup, Denmark) in PBS. Then, the cells were incubated with Alexa Fluor 647-conjugated anti-BrdU (mAb) (Invitrogen) for 1 hour at room temperature in the dark. After washing with PBS, the cells were resuspended in 2 μg/mL propidium iodide (PI) (Sigma) in PBS plus RNase, incubated at 37° C. for 30 min in the dark, and then analyzed by FACSCalibur flow cytometer (Becton-Dickinson, San Jose, Calif., USA).

### Western Blot

Cells were washed with cold PBS and lysed in RIPA buffer (Cell Signaling Technology). After centrifugation at 14,000 rpm for 15 minutes at 4° C., the supernatants were collected and the protein concentrations were measured using BCA protein assay reagent (BIO-RAD). Subsequently, equal amounts of proteins were separated in NuPAGE 4-12% Bis-Tris gradient gel (Invitrogen #NP0335) and transferred onto nitrocellulose membranes (Invitrogen #B301002). After blocking with 5% milk, the membranes were then probed at 4° C. overnight with various primary antibodies: anti-γ-H2AX (Cell Signaling), anti-phospho-Chk1 (Ser345) (Cell Signaling), anti-phospho-Chk2 (Thr68) (Cell Signaling), anti-PRMT5 (Abcam), anti-RPA1 (Abcam), anti-phospho-RPA2 (S4/S8) (Bethyl Laboratories), anti-RPA2 (Ab-2) (Calbiochem), cleaved caspase-3 (Cell Signaling), and anti-β-actin (Sigma); washed with TBST (20 mM Tris, 150 mM NaCl, 0.1% polysorbate 20; pH 7.6); and incubated with horseradish peroxidase (HRP)-conjugated secondary antibodies (Promega) at room temperature for 1 hour. Finally, after washing with TBST, the antibody-bound membranes were treated with enhanced chemiluminescent Western blot detection reagents (GE Healthcare) and visualized with an x-ray film (GE Healthcare).

### Immunofluorescence Staining

Cells grown on glass coverslips (VWR) were rinsed with PBS, and then fixed in 4% formaldehyde for 15 min. The cells were subsequently treated with 0.2% Triton X-100 in PBS for 10 min. After blocking with 2% BSA in PBS containing 5% FBS at RT for 30 minutes, cells were incubated with an appropriate primary antibody γ-H2AX (Cell Signaling) for 2 hours. Then the cells were washed with PBS and incubated for 1 hour with secondary antibody [Alexa Fluor-488 goat anti-mouse immunoglobulin G (IgG) (H+L) conjugate or anti-rabbit IgG (H+L) conjugate (Invitrogen)]. After washing with PBS, the coverslips were dried, and then reversely covered onto slides (Fisher Scientific) by adding mounting medium with 4′,6-diamidino-2-phenylindole dihydrochloride (DAPI) (Vector Laboratories). A LSM-710 confocal microscope (Zeiss) was used to obtain fluorescence images.

### Comet Assay

The comet assay measures DNA damage in individual cells. It was performed according to the instructions of the OxiSelect Comet Assay Kit (Cell Biolabs). Briefly, microscope slides were first covered with a normal melting point agarose to create a base layer. Then, 1-2×10⁵ cells were embedded into 75 μL of low-melting-point agarose at 37° C. and the gel was cast over the first agarose layer. Then slides were immersed into a lysis buffer and kept for 1 hour at 4° C. After cell lysis, the slides were electrophoresed in alkaline electrophoresis buffer (300 mM NaOH, 1 mM EDTA, pH 13). The slides were then stained with Vista Green DNA dye. Comet tails were measured using Image J.

### NHEJ and HR Repair Assays

NHEJ and HR assays to examine the repair efficiency of I-SceI inducible-double strand breaks (DSBs) were performed using NHEJ/DsRed293B and HeLa DR13-9 cell lines, respectively, as previously described with slight modifications. Briefly, 3×10⁵ cells were plated on 6-well dishes and 2 μg I-SceI expression vector pCPASce was transfected using Lipofectamine2000 (Invitrogen). 24 hours post-transfection, the indicated amount of gemcitabine and/or EPZ015666 was incubated with cells for 24 hours. The DsRed- and GFP-expressing cells were counted in flow cytometric analysis (BD FACS Calibur and CellQuest Pro) by the FL2 and FL1 channels for NHEJ and HR repair efficiency, respectively. The % of fluorescent positive cells in the treatment of gemcitabine and/or EPZ015666 was normalized to that of the non-treatment cells (Ctrl) transfected with pCPASce to calculate the relative repair efficiency.

### RPA Overexpression

WT RPA1/2/3 and GFP were overexpressed in the PRMT5 KO cell line by co-transfection of WT RPA1/2/3 (Addgene) and pCMV-GFP plasmid with 5:1 ratio. The wild-type cell line was transfected with pCMV-GFP plasmid alone as a control. 10 μg total DNA was used in the presence of 30 μL of Fugene6 reagent in 10 cm plate dish that had 70% confluency. 24 hours after transfection, GFP positive cells were sorted by a FACS Aria cell sorter.

### In Vivo Xenograft Experiments

All animal care and experimental procedures were carried out in accordance with protocols approved by the University of Virginia School of Medicine Animal Care and Use Committee. To develop xenograft tumors, Control sgRNA infected WT cells and PRMT5-KO cells were subcutaneously injected into the dorsal flanks of 8 week-old nude mice, which were obtained from the Jackson Laboratory (Bar Harbor, Me., USA). When the tumors were visible (approximately 30 mm³ in volume), the mice received respective gemcitabine treatments via intraperitoneal (i.p.) injection. After weekly monitoring, time to appearance of the tumor was recorded, and the tumor volume was measured by caliper. The tumor volume was calculated as follows: volume=longest tumor diameter×(shortest tumor diameter) 2/2. After 35 days of treatment, the mice were euthanized by CO₂ inhalation, and the tumor tissues were collected for further analyses.

### RNA-Seq and Library Preparation

Control sgRNA (CgRNA) and PRMT5-KO cells were treated with gemcitabine (200 nM) or EPZ015666 (500 nM) for 48 hours. Total RNA was purified using an RNeasy mini kit (Qiagen #74104) by following the kit instructions. mRNA was isolated by using NEBNext Poly(A) mRNA Magnetic Isolation Module (New England Biolabs #7490S). RNA-Seq libraries were prepared using the NEBNext Ultra Directional RNA Library Prep Kit for Illumina (New England Biolabs #E7420S) by following the company's protocol. A Qubit measurement and bioanalyzer were used to determine the library quality.

### Data Analysis for RNA-Seq

General sequencing data quality was examined using FastQC (v. 0.11.5). RNA-Seq data were aligned to the human reference genome (hg19) using HISAT2 (v. 2.1.0) with the default paired-end mode settings. The resulting sam files were sorted by reading names and converted to bam files using samtools (v. 1.9) sort command. For ATAC-Seq, sequencing reads mapped to mitochondria DNA were removed from the bam files using the samtools view. Then the bam files were sorted by mapping position and indexed using corresponding samtools commands. The sorted and indexed bam files were first converted to bigwig files for visualization in the UCSC Genome Browser to avoid technical alignment errors. Next, the bam files were quantified against gencode (v27lift37) annotation using Stringtie (v. 1.3.4d) with the default settings.

After obtaining the gene count matrix from Stringtie, we imported it into R and normalized the data following the pipeline of DESeq2. Specifically, to ensure a roughly equal distribution for all the genes across samples, we used rlog transformation to stabilize expression variance for genes with different expression levels. Then samples were clustered according to Euclidean/Poisson distances to make sure replicates are clustered together. By calling the DESeq function, we determined genes with significant expression changes between the PRMT5 WT and KO samples thresholding at an adjusted P value of 0.01. Heatmaps were produced using the pheatmap R package. All other plots were generated using ggplot2. Gene set enrichment analysis (Subramanian, et al. 2005) were performed using the GSEA website and the stand-alone GSEA program referencing the Molecular Signatures Database (MSigDB).

Data Analysis for Three Publicly Available PDAC Studies from GEO and TCGA

Data were downloaded from the Gene Expression Omnibus (GEO) database for publicly-available studies. Normalized PRMT5 expression was compared between relevant sample groups using appropriate student's t-test. Analyses were performed, and plots were generated in RStudio v. 0.99.484 with R v. 3.3.0. The survival analysis for PDAC patients with high (>0.5 standard deviation [s.d.]) and low expression (<0.5 s.d.) of PRMT5 was carried out through cBioPortal.

### Example 2: In Vivo CRISPR Gene KO Screening

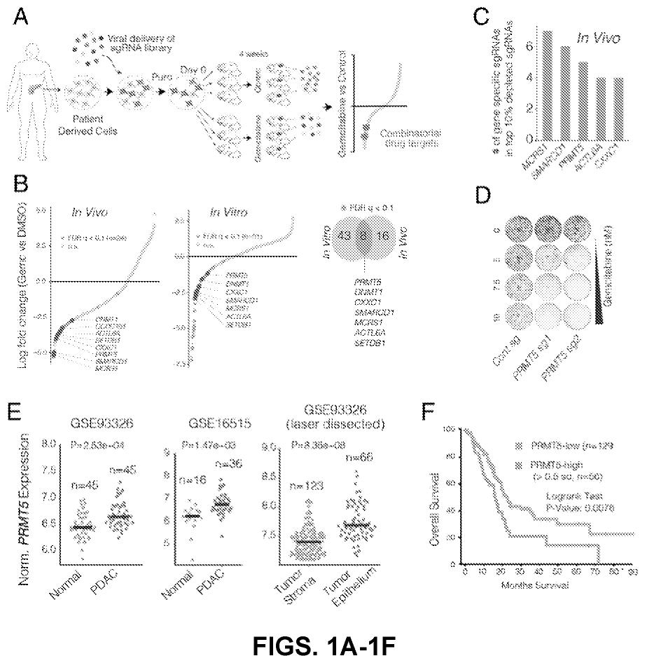

We performed the CRISPR screening using a clinically-relevant patient-derived xenograft (PDX) model of PDAC in which a patient's tumor is propagated in vivo within the pancreas of athymic nude mice. The PDX366 line is established from a poorly-differentiated metastatic tumor with low stromal content and mutant for KRAS, P53, and SMAD4 but the wild type (WT) for P16 genes. In our CRISPR screen (FIG. 1A), we used an 8,031 single-guide RNA (sgRNA) library targeting 619 human genes enriched for chromatin modifiers plus 360 control sgRNAs.

To maintain sgRNA coverage, we infected ˜50-100 million cells at ˜0.25 multiplicity of infection (MOI). After a week of drug selection, the surviving cells were randomly divided into 9 batches, each containing ˜2 million cells (˜200× sgRNA coverage). Of these, one sample was harvested as “day 0,” and others were maintained in culture for in vitro screening or for xenograft injection into the pancreas of athymic nude mice (˜2 million cells/mouse, 6 mice total). One week after injection, animals were randomized to receive either vehicle control (n=3) or gemcitabine treatment (n=3) for 4 weeks (see FIG. 1A). The relative abundance of each sgRNA was assessed by targeted amplification and deep sequencing of tumor genomic DNA. Data analysis was performed using MAGeCK and R. In parallel, we also performed in vitro screening, in which cultured cells were exposed to control dimethyl sulfoxide (DMSO) or 20% inhibitory concentration (IC20) doses of Gem every 3 days for 4 weeks.

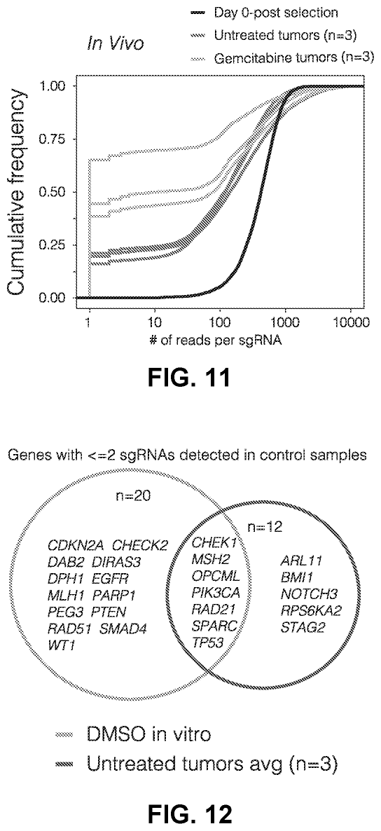

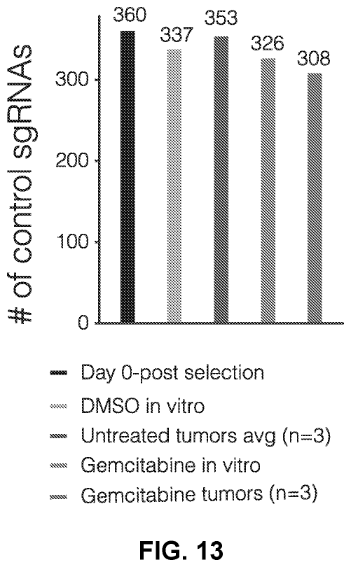

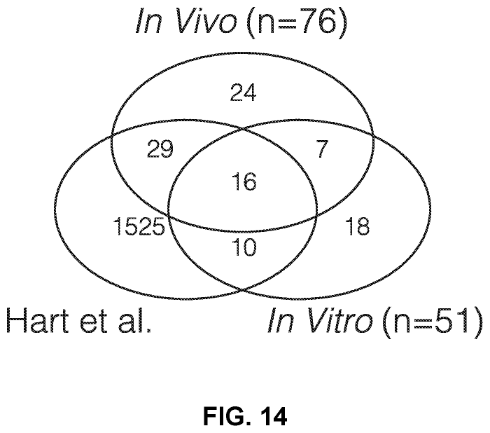

The sgRNA read count distribution analyses of the day 0 sample (>99.9% coverage) demonstrated that the sgRNAs in our library were evenly represented with a Gini index of 0.07 (˜0.1 is suggested for initial-state samples, FIG. 10). Contrary to the day 0 samples, 86%, and 81% of the sgRNAs were detectable in control in vitro and in vivo samples after a month of selection, respectively. Assuming that the ˜15% depletion was due to the functional roles of the target genes, the analyses suggested that ˜95% of cells containing sgRNAs contributed to in vivo tumor formation (FIG. 11). Notably, only 20 and 12 genes had 2 or fewer sgRNAs in the in vitro and in vivo control samples, and 7 genes were overlapped (FIG. 12). The 7 genes include essential DNA repair genes like CHEK1, MSH2, and RAD21 (FIG. 12). One of the in vivo Gem treated tumors had substantially more sgRNAs depleted compared to the other two replicates. Reasoning that this tumor responded to gemcitabine at a higher than expected rate, we excluded it from the downstream analysis (FIG. 11). Since the non-genomic targeting control sgRNAs were well-represented in all of the samples, they were used to profile the null distribution of Robust Rank Aggregation (RRA) scores when calculating the P values (FIG. 13). Negative selection RRA scores identified genes that were consistently depleted when compared to day 0 samples, indicating that these genes are critical for the survival of the PDX cell line (FIG. 14). To check this, we compared these set of genes with known essential fitness functions. Critically, more than half of the 104 critical survival genes that we identified from the in vitro and in vivo samples overlapped with the previously identified essential gene list from five independent cell lines. It is also notable that nearly ⅓ of essential fitness genes we identified are in vitro or in vivo specific, indicating their differential essentiality for different growth conditions. Reasoning the limited therapeutic index of targeting these essential genes, we excluded them from the candidate genes that showed synthetic lethality with Gem.

### Example 3: Identifying Genes Whose Depletion Results in Synthetic Lethality with Gemcitabine

We aimed to identify genes that could be therapeutically targeted to synergistically boost the therapeutic effect of Gem. We, therefore, prioritized our CRISPR screening hits based on three criteria. Firstly, the gene must have been significantly depleted both in vitro and in vivo. We also included the in vitro screening data so that we could robustly validate the screening hits using in vitro assays. Secondly, the potential hit must have been “druggable,” i.e., have an existing small molecule inhibitor. And finally, targeting the CRISPR hit should have a high potential for strong therapeutic value. Among these three criteria, the latter one is more ambiguous. To this end, we focused on genes whose high expression has strong negative prognostic value for PDAC patients.

This primary screening (FIG. 1A) identified MCRS1, SMARCD1, PRMT5, CXXC1, SETDB1, ACTL6A, and DNMT1 as significant hits whose depletion was potentially lethal with Gem (FIGS. 1B-C). We then performed a validation screening with additional sgRNAs for each of these genes using the PDX366 cell line. PRMT5 scored as the top hit whose depletion synergistically increased Gem cytotoxicity (FIG. 1D). PRMT5 is the primary type II PRMT that is responsible for the majority of symmetric demethylation (SDMA) on the arginine residues of its targets, which include various histone proteins as well as transcription factors. PRMT5 is implicated in diverse functions, including genome organization, transcription, cell cycle, and spliceosome assembly. The role of PRMT5 in the proliferation of cancer cells is increasingly appreciated. Importantly, PRMT5 is a druggable protein with several selective inhibitors available and many of which are currently tested in clinical trials (e.g., NCT03573310 and NCT03854227). However, its significance in PDAC progression or its potential as a combinatorial therapeutic target in PDAC cells has not been explored.

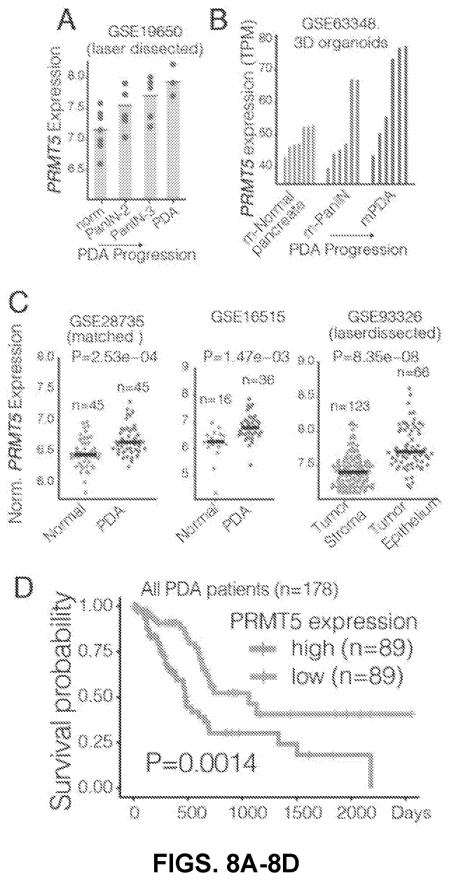

Initial pathological observations followed by experimental validation in genetic mouse models show that PDAC progression is a multi-step process where the driver genetic mutations, such as oncogenic KRAS mutations, transform normal ductal cells into pancreatic intraepithelial neoplasia (PanIN). Additional loss of function mutations in tumor suppressor genes such as SMAD4 and TP53 result in progression of PanINs into infiltrating pancreatic ductal adenocarcinoma (PDAC). The progressive PDAC development has been modeled in genetic mouse models and a 3D organoid system. Our analysis of laser dissected cells from normal human duct cells, PanINs, and PDAC cells suggests that PRMT5 expression is progressively increased during human PDAC pathology (FIG. 8A). This is further supported by expression data from the mouse organoid system (FIG. 8B).

We then determined whether PMRT5 expression is aberrantly and selectively upregulated in pancreatic cancer cells compared to adjacent stromal cells. To this end, we analyzed gene expression data from multiple public resources including normal-matched PDAC tumor microarray data, tumor-adjacent normal versus PDAC tumor, and laser microdissected PDAC tumor cells versus adjacent stromal cells. Importantly, this unbiased analysis of independently generated data sets showed that PRMT5 mRNA expression is significantly upregulated in PDAC cells versus normal stromal cells (FIG. 8C). Most critically, the analysis of The Cancer Genome Atlas (TCGA) PDAC patient data shows that tumors with high PRMT5 expression result in significantly shorter overall patient survival (FIG. 8D), indicating that PRMT5 is a critical component of PDAC progression and therapy response and thus a promising therapeutic target.

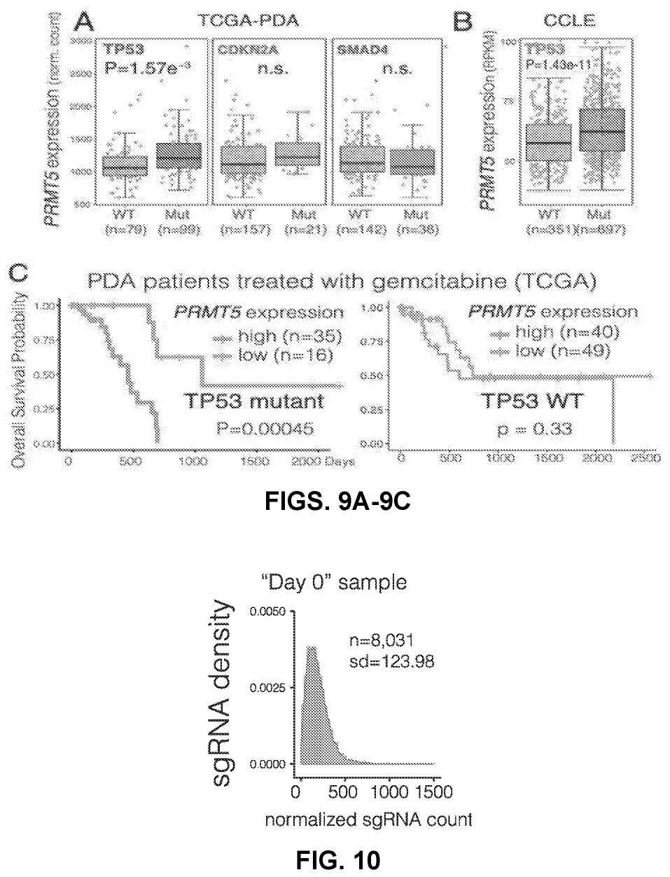

Since PDAC tumors have higher PRMT5 expression compared to stromal cells, targeting PRMT5 should have a therapeutic value because PRMT5-low expressing patients have significantly longer survival. However, a large degree of inter-tumor PRMT5-expression heterogeneity exists. It is thus also important to understand whether PRMT5 is differentially expressed in cancers with certain genetic makeups. A computational approach was used to exploit PDAC genomic data in TCGA and the Broad Institute Cancer Cell Line Encyclopedia (CCLE) data sets. We specifically explored whether PRMT5 expression is differentially regulated due to a recurrent PDAC-specific genetic alteration. Since most PDAC tumors harbor oncogenic KRAS mutations, we studied PRMT5 expression in recurrently mutated tumor suppressor genes in PDAC including CDKN2A, TP53, and SMAD4, which are mutated in about 90, about 80, and about 55 percent of PDAC tumors, respectively. We initially analyzed RNA-seq expression data from 189 PDAC tumors in TCGA data. Only TP53 mutant tumors have significantly higher PRMT5 expression compared to WT tumors (FIG. 9A). Our analysis of >1000 CCLE cancer cell expression data sets is in line with PDAC data, suggesting that PRMT5 expression is higher in TP53 mutant cancer cells (FIG. 9B).

Critically, the chemotherapy treatment regimen is known for some of the TCGA PDAC patients. Based on preliminary results, we analyzed the TCGA data set to test our overall hypothesis that gemcitabine treatment of PRMT5-low tumors will have more cytotoxicity and thus more favorable patient outcomes. Among the PDAC patients that received gemcitabine, patients with low PRMT5 expressing TP53 mutant tumors survived significantly longer than PRMT5 high expressing patients, who generally had an extremely poor prognosis (p=0.00045, FIG. 9C). Furthermore, this correlation was completely absent in TP53 WT tumors, regardless of PRMT5 levels (FIG. 9C, right panel).

We therefore investigated the potential role of PRMT5 in PDAC progression and aimed to assess whether PRMT5 inhibition has a potential therapeutic value for PDAC. To this end, we initially analyzed whether PRMT5 expression is differentially regulated in PDAC tumors and has a prognostic value for the survival of patients. The gene expression analysis of multiple independently generated datasets such as normal-matched PDAC tumor, tumor-adjacent normal vs PDAC tumor, and laser microdissected PDAC tumor cells vs. adjacent stromal cells shows that PRMT5 mRNA expression is significantly upregulated in PDAC cancer cells compared to normal stromal cells (FIG. 1E). Most critically, the analysis of TCGA PDAC patient data shows that tumors with high PRMT5 expression result in significantly shorter overall patient survival (FIG. 1F), indicating that PRMT5 is a critical player in PDAC progression or therapy response, and thus a promising therapeutic target.

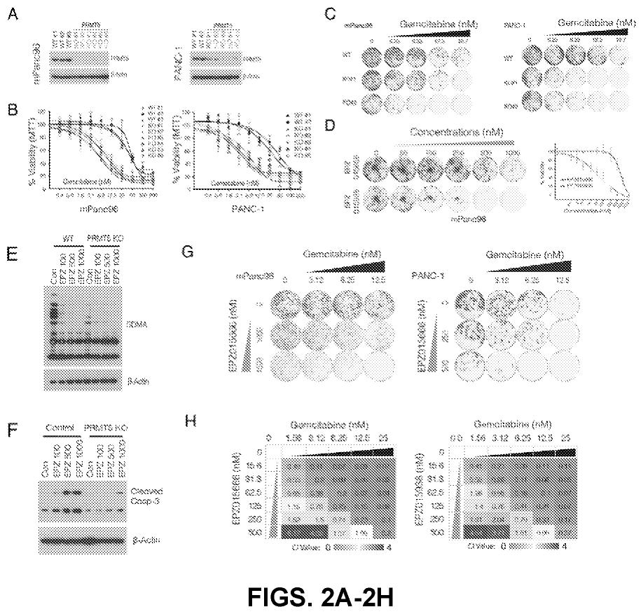

To better study the role of PRMT5 in the cellular response to Gem, we generated multiple single KO clones in two additional pancreatic cancer cell lines (mPanc96 and PANC-1) (FIG. 2A). Notably, despite screening for ˜100 single clones, we seldom observed full depletion of PRMT5 at the protein level, especially in PANC-1 cells. However, the clones with even partial PRMT5 depletion were nearly an order of magnitude more sensitive to Gem compared to wild-type (WT) clones (4-5 μM vs. ˜50 μM IC₅₀) as measured by cell viability assay (FIG. 2B). In line with these, longer-term crystal-violet colony formation assays also demonstrated that the PRMT5 KO clones were significantly more sensitive to Gem compared to WT cells (FIG. 2C).

To further corroborate these genetic depletion results, we tested two separate small molecule pharmacological inhibitors (EPZ015666 & EPZ015938) that specifically target PRMT5. When tested as a single agent, EPZ015938 had substantially more growth inhibition activity on colony formation (FIG. 2D). As anticipated, EPZ015666 treatment significantly inhibits global SDMA (FIG. 2E). Furthermore, the inhibitor is specific towards PRMT5 as it results in significant apoptosis (caspase-3 cleavage) selectively in WT cells but not in PRMT KO cells (FIG. 2F). These inhibitors significantly potentiated Gem growth inhibition activity at multiple dose combinations as measured by long-term colony formation assay (FIG. 2G). To better assess whether PRMT5 inhibitors are synergistic with Gem, we calculated the Combination Index (CI) values for each dose combination. The CI<1 indicates synergy between two drugs, whereas CI≈1 is additive, and CI≥1.2 suggests an antagonistic effect. Importantly, of the 24 dose combinations for two separate inhibitors, we observed robust synergistic activity for ˜80% of EPZ015666+Gem and ˜78% of EPZ015938+Gem dose combinations (FIG. 2H).

### Example 4: Understanding Pathways Underlying PRMT5 Depletion-Mediated Vulnerability to Gem

Encouraged by the genetic depletion and pharmacological inhibition studies, we then aimed to understand the molecular mechanism of conditional sensitivity to PRMT5 depleted cells to Gem and assess the therapeutic value of this combination in vivo. At the chemical level, Gem is composed of di-fluoro-deoxycytidine (dFdC). Mechanistically, it exerts its biological effects by inducing replication stress in fast-dividing cancer cells. Once taken up by the cells, dFdC is converted into dFdC-diphosphate (dFdCDP) and dFdC-triphosphate (dFdCTP). dFdCTP incorporates into DNA as a cytosine analog and blocks DNA synthesis due to strand termination. Additionally, dFdCDP also inhibits the ribonucleotide reductase enzyme, thereby resulting in depletion of the dNTP pool necessary for DNA synthesis.

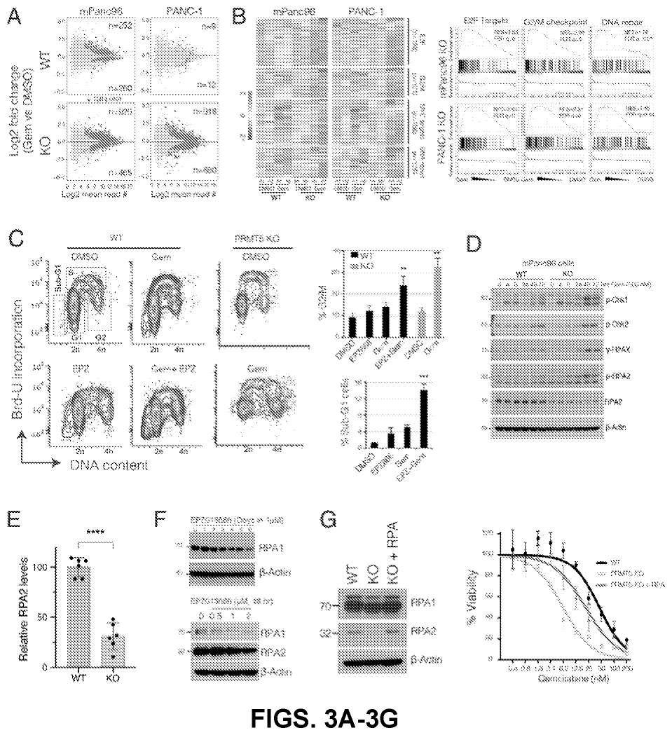

Since PRMT5 is a major transcriptional regulator, we initially investigated whether PRMT5 depleted cells had a differential transcriptional response to Gem. We therefore comparatively analyzed the transcriptional responses of PRMT5 WT and KO cells to Gem in two independent PDAC cancer cell lines. Critically, the KO cells responded to Gem by differentially regulating a much larger number of genes. For example, while only 21 genes (9 up, 12 down) in WT PANC-1 cells and 512 genes (252 up, 260) in mPanc96 WT cells were significantly altered, 1,598 genes (918 up, 680 down) in PANC-1 KO cells and 1,385 genes (920 up, 465 down) in the mPanc96 KO cells were significantly altered in response to treatment with IC₃₀ Gem for 24 hours (FIG. 3A). These results suggested that physiological levels of PRMT5 are required to buffer global transcriptional response to Gem. Comparative gene set enrichment analysis demonstrated that genes implicated in cell cycle, and DNA repair pathways were aberrantly active in the KO cells when treated with Gem (FIG. 3B). Gene sets identifying cell cycle-related genes such as G2/M checkpoints, and E2F and MYC targets were all more strongly upregulated in Gem treated KO cells compared to WT cells. Furthermore, genes involved in DNA repair were among the most highly differentially regulated genes when the KO cells were treated with Gem (FIG. 3B).

These results support the hypothesis that PRMT5 depletion-mediated conditional vulnerability to Gem was partially due to the aberrant regulation of cell cycle and DNA repair pathways. To test this hypothesis, we set out several molecular assays to study the mechanism of PRMT5 depletion mediated aberrant cell cycle and DNA repair programs. The analysis of cell cycle position through BrdU incorporation showed that Gem treatment of WT cells resulted in a partial delay in cell cycle with a substantial accumulation of cells in S-phase and partial increases in G2/M cells (FIG. 3C). On the other hand, the combination of Gem and PRMT5 inhibitor resulted in a significant accumulation of G2/M cells and sub-G1 dead cells (FIG. 3C). In line with the pharmacological inhibition of PRMT5, Gem treatment resulted in a significantly higher number of G2/M cells in the PRMT5 KO cells compared to WT cells.

### Example 5: PRMT5 Depletion Results in RPA Exhaustion

Coordinated activation of cell cycle checkpoints and cell cycle arrest is one of the primary mechanisms that enable cells sufficient time to repair DNA against external cues. The robust arrest of cells at the G2/M cell cycle led us to study the activation of checkpoints further. The S and G2/M cell cycle arrest results from DNA damage that mediates activation of ATR-Chk1-Cdc25C. We, therefore, performed time-course experiments to study whether Gem treatment resulted in differential activation of DNA damage and cell cycle checkpoints in the KO cells. Notably, we detected sustained and stronger phospho-Chk1 (a marker of DNA damage, S and/or G2/M arrest), γ-H2AX (a marker of DNA damage) as well as phospho-RPA2 (a marker of DNA damage and replication stress) in the KO cells compared to WT cells (FIG. 3D).

This analysis also revealed something unexpected to us. Although we observed a strong induction of phospho-RPA2 in the KO cells, the total levels of RPA2 were substantially lower in the KO cells (FIG. 3D). Further quantitative analyses suggested that the depletion of PRMT5 resulted in a significant reduction in RPA2 protein levels (p<0.0001). We observe a ˜60-70% reduction in RPA2 levels in the PRMT5 KO cells compared to WT cells (FIG. 3E). These findings led us to investigate whether the depletion of RPA was due to enzymatic activity of PRMT5. Critically, both time-course, as well as dose-escalation experiments, showed that depletion of PRMT5 activity through small molecule inhibitors resulted in RPA2 depletion (FIG. 3F and FIG. 7).

It should be noted that RPA2 is one of the three subunits of the RPA complex, which is viewed as the guardian of the genome, because it binds and protects any single-stranded DNA that forms during DNA replication, transcription and repair pathways. The cytotoxicity of Gem in fast-dividing cancer cells is mostly due to the creation of replication stress (RS) by blocking DNA synthesis and diminishing the dNTP pool by inhibiting ribonucleotide reductase enzyme. During replication stress, RPA becomes essential to protect single-strand DNA (ssDNA) at the stalled replication forks. Critically, overall RPA levels are crucial determinants as to whether cells can resolve the stalled forks. In low RPA conditions, the replication stress leads to “replication catastrophe,” where chromosomes chatter with thousands of double-strand breaks (DSB). These findings lead to the “RPA exhaustion” hypothesis, which states that when RPA is not sufficient, cells can't survive the replication stress, and the stalled replication forks collapse, which results in the breakage of forks, and ultimately replication catastrophe.

Our results so far support the hypothesis that PRMT5 depletion results in “RPA exhaustion,” and therefore, cells are not able to cope with Gem-mediated DNA damage. To test this hypothesis, we aimed to replenish the RPA complex to see if it could rescue the PRMT5 depletion phenotype. Since RPA works as a tri-partite complex where each subunit is needed at an equimolar ratio, we exogenously provided cells with a vector that expresses all three subunits. Importantly, replenishing the RPA complex in PRMT5 KO cells to near equal levels to WT cells (FIG. 3G) results in significant resistance to Gem in PRMT5 KO cells. These results suggest that, at the molecular level, the PRMT5 depletion-mediated Gem-sensitivity phenotype is, in part, due to exhaustion of the RPA complex.

### Example 6: PRMT5 Depletion Results in Excessive DNA Damage Accumulation

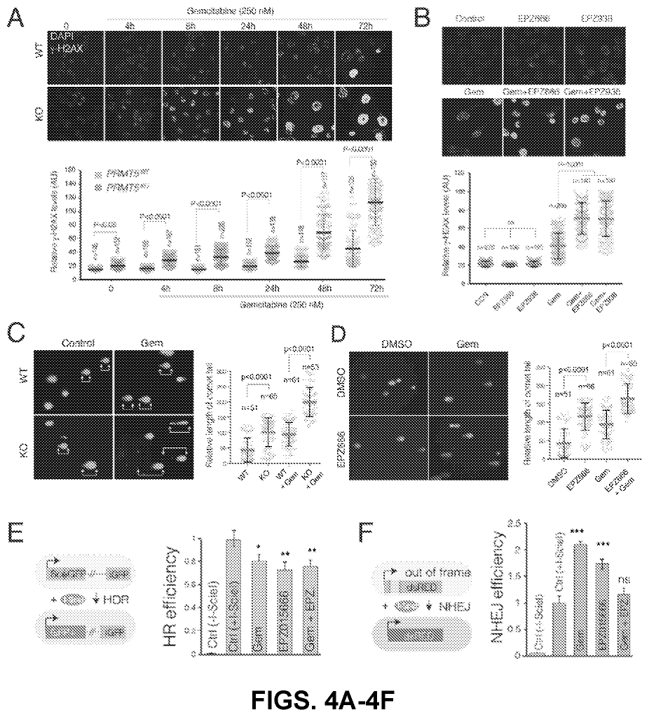

Our expression analysis also highlighted that genes involved in DNA repair pathways were aberrantly regulated when PRMT5 KO cells were treated with Gem. Furthermore, the above results suggested that due to RPA exhaustion, PRMT5 depleted cells are not able to resolve a stalled replication fork, which may result in the collapse of the fork and accumulation of DNA DSB. We, therefore, utilized two independent molecular assays to detect and quantify Gem-induced DNA damage in control and PRMT5 depleted cells. Initially, we used immunofluorescence (IF) assays to detect the phosphorylated H2AX (γ-H2AX), which is a modified histone variant deposited into an around mega-base chromatin region around DSB. Critically, strong γ-H2AX foci can be detected as early as 4 hours post Gem treatment in the KO cells. On the other hand, it took 48-72 hours to detect similar levels of γ-H2AX foci in WT cells using the same concentration of Gem (FIG. 4A). Quantitative analysis of γ-H2AX foci formation levels over a period of 72 hours showed that PRMT5 KO cells consistently had significantly higher levels of γ-H2AX, indicating higher levels of DNA DSB due to Gem treatment (FIG. 4A, lower panel, bar plots). In line with the genetic depletion of PRMT5, pharmacological inhibition of PRMT5 with two separate small molecule inhibitors also demonstrated that depletion of PRMT5 activity resulted in significant accumulation of DNA DSB, as detected by levels of γ-H2AX foci formation (FIG. 4B). In addition to γ-H2AX foci formation, we also measured the level of DNA strand breaks through a comet assay, which measures the overall levels of DNA damage, as done through single-cell gel electrophoresis. As the frequency of DNA breaks increases, so does the fraction of the DNA extending towards the anode, forming the comet tail. The length of the tail is an indication of levels of fragmented DNA in individual cells. In line with the γ-H2AX IF results, we observed a significantly longer comet tail when PRMT5 KO cells or PRMT5-inhibited cells were treated with Gem compared to WT and control-treated cells, respectively (FIGS. 4C-D).

### Example 7: PRMT5 Depletion Results in Impaired NHEJ

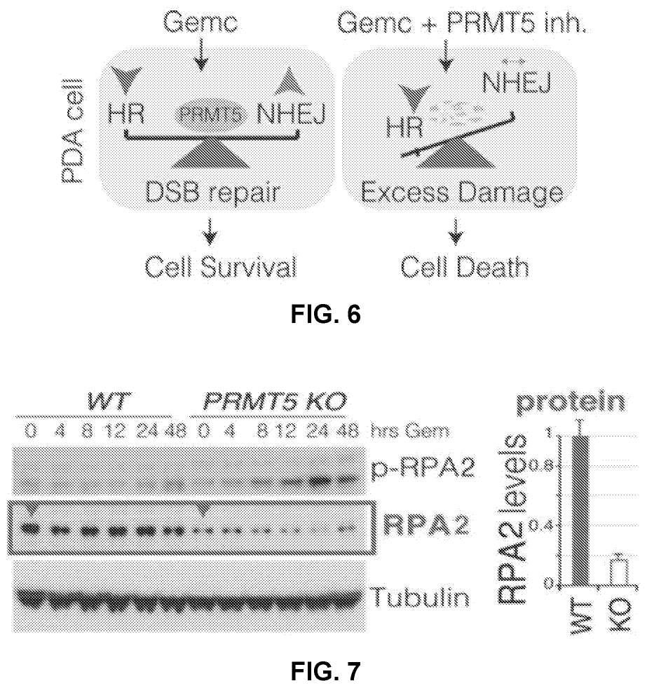

Depending on the time and kind of DNA damage, DSB is repaired through either precise homology-directed DNA repair (HDR) or error-prone NHEJ. NHEJ is active throughout the cell cycle, whereas HDR is restricted to the late S and G2 phases of growing cells. Our differential gene expression results, as well as RPA exhaustion findings, led us to investigate whether excessive DNA damage accumulation was, in part, due to impaired DNA repair activity. To this end, we used I-SceI endonuclease-based genetic reporters where relative efficiency of DSB repair by either pathway could be robustly quantified. In the NHEJ reporter assay, the dsRED contains a “stuffer sequence” flanked by two I-SceI recognition sites, which puts dsRED out of frame. On the other hand, for the HDR GFP-reporter system, the construct contains two defective GFP genes, the first one contains an I-SceI site. In both cases, the engineered HeLa cells are dsRed (−) or GFP(−), respectively. However, exogenous expression of I-SceI leads to a DSB repair and creation of either dsRED+ or GFP+ cells, which can be quantified to assess relative NHEJ or HDR repair efficiencies by quantifying the percentage of dsRED+ or GFP+ cells upon I-SceI expression. Our results show that either Gem or PRMT5 inhibitor treatment significantly inhibits the HDR activity (FIG. 4E). Notably, the combination treatment did not result in any further reduction in HDR activity, suggesting that the reduced HDR activity did not explain the observed synergistic accumulation of DNA damage. We then assessed whether PRMT5 inhibitor alone or in combination with Gem results in differential NHEJ activity. Importantly, unlike HDR activity, we observed a significant increase in NHEJ activity when cells were treated with either Gem or PRMT5 inhibitor. Surprisingly, when cells were treated with the Gem plus PRMT5 inhibitor combination, there was no significant change in NHEJ activity (FIG. 4F). This finding supports a hypothesis that reduced HDR repair due to a single Gem or PRMT5 in treatments is compensated by an increase in NHEJ (see FIG. 6). However, the combination treatment was not able to increase the NHEJ repair pathway and thus cannot compensate for the reduced HDR activity.

### Example 8: Combinatorial Treatment Results in Synergistic Tumor Growth Inhibition In Vivo

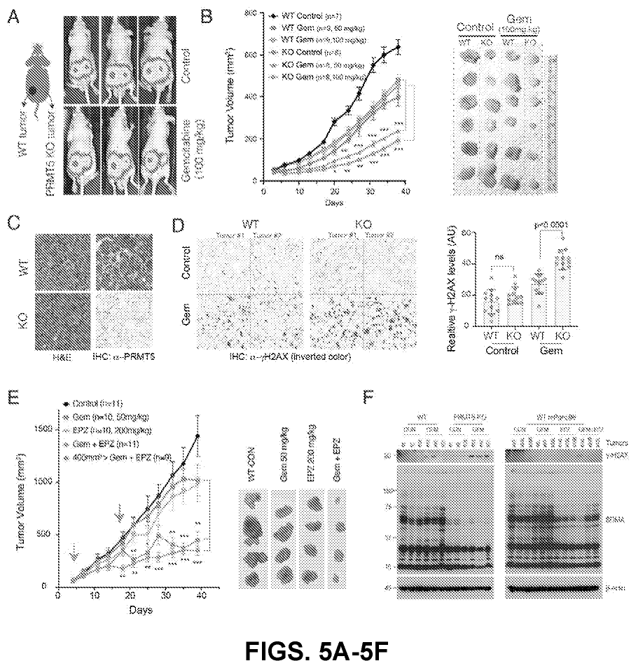

Next, we investigated to see if the combination would result in synergistic growth inhibition of PDAC tumors. To this end, we explored both genetic depletion and pharmacological inhibition of PRMT5 in a xenograft model of PDAC. Initially, we tested whether tumors formed by WT and PRMT5 KO cells were differentially sensitive to Gem treatment. To be able to better compare the tumors from these two genetic backgrounds, we injected 5×10⁵ WT mPanc96 cells in the left flank and the same number of the PRMT5 KO cells in the right flank of the same mouse. This strategy enabled us to compare the two tumors grown in the same mouse. After one week of tumor formation, the mice were randomly divided into three groups where one received control, and the other two received two separate Gem doses (50 mg/kg or 100 mg/kg). Notably, the PRMT5 KO cells were able to form tumors. However, these tumors were slightly smaller than the tumors formed by WT cells. The Gem treatments did result in a notable reduction of WT tumors. However, the most significant reduction of tumor volumes was observed in the PRMT5 KO tumors treated with Gem (FIGS. 5A-B). Starting from the fifth treatment (day 20 of tumor formation), the Gem treated PRMT5 KO tumors were significantly smaller than their WT counterparts or the untreated KO tumors.

We also extracted tumors to analyze their morphology and molecular structure through H&E and IHC for selected markers. The H&E staining of WT and PRMT5 KO tumors demonstrated comparable cellular architecture (FIG. 5C). The IHC staining confirmed that the tumors from PRMT5 KO cells did not express PRMT5 protein (FIG. 5C). We then performed IHC to investigate whether the Gem treatment resulted in greater DNA damage in vivo than was seen in in vitro experiments. Consistent with the in vitro experiments, we observed significantly more γ-H2AX staining in the PRMT5 KO tumors when treated with Gem (FIG. 5D), indicating that Gem resulted in a significantly higher amount of DNA damage in the PRMT5 KO tumors than in the WT tumors.

We next performed in vivo xenograft experiments to assess whether pharmacological inhibition of PRMT5 would result in a synergistic reduction of PDAC tumors in vivo when combined with Gem. To this end, we designed two separate strategies: early treatment and delayed treatment. One set of tumors were treated with control, single-agent drugs or combination of Gem and PRMT5 inhibitor as soon as the tumors reached ˜100 mm³. Importantly, as soon as a week after treatment, we observed a statistically significant reduction in tumor volume only in the tumors receiving the combination treatment. It is notable that the therapeutic effect of PRMT5 inhibitor plus Gem is much stronger than the Gem or PRMT5 inhibitor treatment alone (FIG. 5E). Importantly, we also allowed a set of tumors to grow to significant sizes (˜400 mm³) before starting the combination treatment. Interestingly, we observed a notable decrease in tumor volume after just one cycle of combined treatment (FIG. 5E). These tumors started to grow for the next couple of treatment cycles but then stopped growing and remained significantly smaller in volume compared to the control or single-agent treated tumors. In the end, these tumors were almost indistinguishable from the tumors that received combination treatment from the beginning.

To assess whether the combination treatment abolished PRMT5 function in vivo, we evaluated the overall levels of the γ-H2AX and the symmetric demethylation of arginine (SDMA) in WT tumors, PRMT5 KO tumors as well as single-agent and combination-treated WT tumors. As anticipated, the PMRT5 KO tumors had significantly lower SDMA levels. In line with genetically depleted tumors, the EPZ015666 treated tumors had lower overall levels of SDMA, indicating that the inhibitor doses that we used resulted in substantial inhibition of PRMT5 function in the tumors in vivo (FIG. 5F). However, the γ-H2AX is significantly activated in either the GEM-treated PRMT5 KO tumors or the combination-treated tumors (FIG. 5F), suggesting that activation of the γ-H2AX may be due to the low level of SDMA.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A method for inhibiting tumor growth in a subject comprising administering to the subject (1) a compound that inhibits the activity of a protein arginine methyl transferase (PRMT) and (2) and a DNA damaging agent.

2. A method for reducing the volume of a tumor in a subject comprising administering to the subject (1) a compound that inhibits the activity of a protein arginine methyl transferase (PRMT) and (2) and a DNA damaging agent.

3. A method for inhibiting the level of symmetric arginine dimethylation, asymmetric arginine dimethylation, or a combination thereof of a protein in tumor cells in a subject comprising administering to the subject (1) a compound that inhibits the activity of a protein arginine methyl transferase (PRMT) and (2) and a DNA damaging agent.

4. A method for increasing the activation level of γ-H2AX in tumor cells in a subject comprising administering to the subject (1) a compound that inhibits the activity of a protein arginine methyl transferase (PRMT) and (2) and a DNA damaging agent.

5. The method in any one of claims 1 to 4, wherein compound inhibits the activity of protein arginine methyl transferase 1 (PRMT1).

6. The method in any one of claims 1 to 4, wherein compound inhibits the activity of protein arginine methyl transferase 5 (PRMT5).

7. The method in any one of claims 1 to 4, wherein compound inhibits the activity of protein arginine methyl transferase 1 and 5.

8. The method in any one of claims 1 to 7, wherein the compound inhibits the activity of protein arginine methyl transferase 1 comprises GSK3368715, AMI-1, RM65, DB75, stilbamidine, alantodapsone, DCLX069, or any combination thereof.

9. The method in any one of claims 1 to 7, wherein the compound inhibits the activity of protein arginine methyl transferase 5, wherein the compound is EPZ015666, EPZ015938, JNJ-64619178, PF-06939999, or any combination thereof.

10. The method in any one of claims 1 to 9, wherein the compound that inhibits the activity of protein arginine methyl transferase is administered at a dosage of from about 0.1 mg to about 10 mg/day.

11. The method in any one of claims 1 to 10, wherein the DNA damaging agent comprises gemcitabine, doxorubicin, cisplatin, carboplatin, oxaliplatin, picoplatin, methotrexate, daunorubicin, 5-fluorouracil, capecitabine, floxuridine, 6-mercaptopurine, 8-azaguanine, fludarabine, cladribine, aminopterin, ralitrexed, etoposide, teniposide, campothecin, doxorubicin, epirubicin, idarubicin, or any combination thereof.

12. The method in any one of claims 1 to 10, wherein the DNA damaging agent is gemcitabine is administered at a dosage of from about 800 mg/m² to about 1,400 mg/m².

13. The method in any one of claims 1 to 10, wherein the compound inhibits the activity of protein arginine methyl transferase 5 (PRMT5) and the DNA damaging agent is gemcitabine.

14. The method in any one of claims 1 to 13, wherein the compound that inhibits the activity of protein arginine methyl transferase is administered prior to the administration of the DNA damaging agent.

15. The method in any one of claims 1 to 14, wherein the tumor comprises a solid tumor.

16. The method in any one of claims 1 to 14, wherein the subject has brain cancer, ovarian cancer, prostate cancer, breast cancer, or pancreatic cancer.

17. The method in any one of claims 1 to 14, wherein the subject has pancreatic ductal adenocarcinoma (PDAC).

18. A kit comprising (1) a compound that inhibits the activity of a protein arginine methyl transferase (PRMT) and (2) and a DNA damaging agent.

19. A pharmaceutical composition comprising (1) a compound that inhibits the activity of a protein arginine methyl transferase (PRMT) and (2) and a DNA damaging agent.
